(19) United States
(12) Patent Application Publication
(10) Pub. No.: US 2010/0267672 A1
(43) Pub. Date: Oct. 21, 2010

(54) NOVEL COMPOUNDS OF REVERSE-TURN MIMETICS, METHOD FOR MANUFACTURING THE SAME AND USE THEREOF

(75) Inventors: Kyung-Yun Jung, Seoul (KR); Jae Uk Chung, Kyunggi-do (KR); Min-Wook Jeong, Seoul (KR); Hee-Kyung Jung, Seoul (KR); Hyun-Ju La, Seoul (KR); Sang-Ho Ma, Seoul (KR); Yong-Sil Lee, Seoul (KR)

Correspondence Address: SEED INTELLECTUAL PROPERTY LAW GROUP PLLC, 701 FIFTH AVE, SUITE 5400, SEATTLE, WA, 98104, US

(73) Assignee: Choongwae Pharma Corporation, Seoul (KR)

(21) Appl. No.: 12/759,854

(22) Filed: Apr. 14, 2010

Related U.S. Application Data
(60) Provisional application No. 61/237,530, filed on Aug. 27, 2009.

(30) Foreign Application Priority Data
Apr. 15, 2009 (KR) 10-2009-0032937

(51) Int. Cl. A61K 31/675; C07D 487/04; A61K 31/53; C07F 9/12; A61P 35/02

(52) U.S. Cl. 514/81; 544/184; 514/243

(57) ABSTRACT

Disclosed are novel reverse turn mimetics based on the framework of pyrazino-triazinone, and the use thereof in the treatment of cancers, particularly, acute myeloid leukemia. A method is also provided for manufacturing the reverse turn mimetics on a mass scale.

### CROSS-REFERENCE TO RELATED APPLICATIONS

This application claims the benefit under 35 U.S.C. §119(d) of Korean Patent Application No. 10-2009-0032937, filed Apr. 15, 2009, and under 35 U.S.C. §119(e) of U.S. Provisional Patent Application No. 61/237,530, filed Aug. 28, 2009, which applications are incorporated herein by reference in their entireties.

### BACKGROUND

1. Technical Field

The present disclosure relates to novel compounds of reverse-turn mimetics, a method for manufacturing the same, and the use thereof in the treatment of diseases, such as acute myeloid leukemia.

2. Description of the Related Art

Random screening of molecules for possible activity as therapeutic agents has been conducted for many years and resulted in a number of important drug discoveries. Recently, non-peptide compounds have been developed which more closely mimic the secondary structure of reverse-turns found in biologically active proteins or peptides. For example, U.S. Pat. No. 5,440,013, and Published PCT Application Nos. WO94/03494, W001/00210A1, and W001/16135A2, all to Kahn, each discloses conformationally constrained, non-peptidic compounds, which mimic the secondary structure of reverse-turns. In addition, U.S. Pat. Nos. 5,929,237 and 6,013,458, both to Kahn, describe conformationally constrained compounds which mimic the secondary structure of reverse-turn regions of biologically active peptides and proteins. The synthesis and identification of conformationally constrained, reverse-turn mimetics and the application thereof to diseases were well reviewed by Obrecht (Advances in Med. Chem., 4, 1-68, 1999).

With the significant advancements in the synthesis and identification of conformationally constrained, reverse-turn mimetics, techniques have been developed and provided for synthesizing and screening library members of small molecules which mimic the secondary structure of peptides, in order to identify bioactive library members. Accordingly, attempts have been made to seek conformationally constrained compounds and highly bioactive compounds which mimic the second structure of reverse turn regions of biologically active peptides and proteins. For instance, reverse turn mimetics, methods for manufacturing the same and bioactivities thereof are disclosed in Published PCT Application Nos. WO 04/093828A2, WO 05/116032A2, and WO 07/139,346A1.

Although a great number of reverse turn mimetics have been manufactured, not many compounds have been found to have high bioactivity. Thus, efforts continue to be made to manufacture compounds applicable to the treatment of diseases such as cancer.

Particularly, efforts have been focused on the development of compounds which strongly block the Wnt signaling pathway to effectively suppress the growth of acute myeloid leukemia (AML) cancer cells known to have an activated Wnt signaling pathway.

Also, there is a need for methods of manufacturing highly bioactive compounds on a mass scale if they are found.

### BRIEF SUMMARY

Described herein are novel bioactive compounds, the use thereof as therapeutic agents or prodrugs for cancer, in particular for acute myeloid leukemia, and a method for manufacturing the same on a mass scale.

Various embodiments provide novel compounds, represented by the following Chemical Formula I:

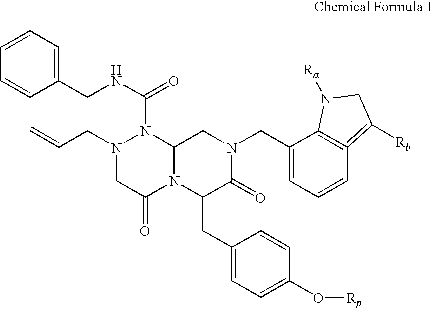

wherein:

Rₐ is a C₁-C₆ alkyl group, a C₂-C₆ alkenyl, or a C₂-C₆ alkynyl group;

R_{b} is an aryl group, a substituted aryl group, or —C(═O)Rₑ wherein Rₑ is a C₁-C₆ alkyl group, a C₂-C₆ alkenyl group, or a C₂-C₆ alkynyl group;

Rₚ, is —H, —PO₃H₂, —HPO₃⁻Na⁺, —PO₃²⁻Na₂⁺, —PO₃²⁻K₂⁺, —PO₃²⁻Mg²⁺, —PO₃²⁻Ca²⁺,

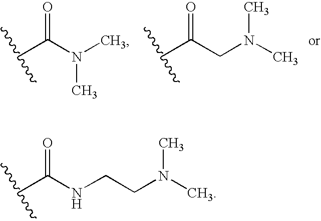

In certain embodiments, a substituted aryl may be an acyl-substituted aryl (as defined herein).

In one embodiment, in Chemical Formula I, Rₐ is a C₁-C₆ alkyl group or a C₂-C₆ alkenyl group; R_{b} is —C(═O)Rₑ wherein Rₑ is C₁-C₆ alkyl; and Rₚ is —H, —PO₃H₂, —HPO₃⁻Na⁺, or —PO₃²⁻Na₂⁺.

In another embodiment, in Chemical Formula I, Rₐ is methyl; R_{b} is —C(═O)Rₑ wherein Rₑ is C₁-C₆ alkyl; and Rₚ is —H.

In yet another embodiment, in Chemical Formula I, Rₐ is methyl; R_{b} is —C(═O)Rₑ wherein Rₑ is C₁-C₆ alkyl; and Rₚ is —PO₃H₂, —HPO₃⁻Na⁺, or —PO₃²⁻Na₂⁺.

In another embodiment, in Chemical Formula I, if Rₐ is methyl and if R_{b} is —C(═O)Rₑ, Rₑ is not methyl or cyclopropyl.

Any compounds specifically disclosed in PCT Application Publication No. WO 09/051,397 (e.g., compounds disclosed in Tables 2-4) are excluded from the scope of the present disclosure.

In one aspect, the present disclosure provides a pharmaceutical composition comprising a compound provided herein and a pharmaceutical acceptable excipient.

In another aspect, the present disclosure provides a method for treating acute myeloid leukemia (AML) comprising administering to a patient having AML an effective amount of the compound or composition provided herein. In certain embodiments, the method comprises injecting an effective amount of the compound or composition to a patient having AML.

In another aspect, the present disclosure provides a method for manufacturing the compound provided herein, comprising the following sequential steps: (a) introducing an acyl group into indole-7-carbaldehyde through Friedel-Crafts Acylation to provide 3-acyl-indole-7-carbaldehyde; (b) introducing an alkyl group and an aminoacetal group to 3-acyl-indole-7-carbaldehyde to provide a 1-alkyl-3-acyl-indole derivative; (c) amidating the 1-alkyl-3-acyl-indole derivative with stereoselectivity Cbz-Tyrosine-OtBu and 2-(1-allyl-4-benzylsemicarbazido)acetic acid to provide a reaction intermediate; (d) cyclizing the reaction intermediate in the presence of formic acid to provide a cyclic intermediate; and (e) phosphorylating the cyclic intermediate to provide a compound of Chemical Formula (I). In certain embodiments, 2-(1-allyl-4-benzylsemicarbazido)acetic acid is synthesized by the following sequential steps: (1) adding TEA (triethylamine) to an ethylhydrazinoacetate solution to provide a reaction solution; (2) adding allyl bromide to the reaction solution; and (3) adding benzylisocyanate. In certain further embodiments, allyl bromide and benzylisocyanate are added in a dropwise manner.

In a related aspect, the present disclosure provides a method for preparing a compound of Chemical Formula (I), comprising: (a) converting indole-7-carbaldehyde to

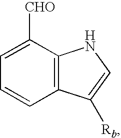

wherein R_{b} is an aryl group, a substituted aryl group, or —C(═O)Rₑ, wherein Rₑ is a C₁-C₆ alkyl group, a C₂-C₆ alkenyl group, or a C₂-C₆ alkynyl group; (b) converting

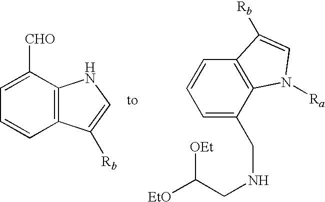

wherein Rₐ is a C₁-C₆ alkyl group, a C₂-C₆ alkenyl, or a C₂-C₆ alkynyl group; (c) amidating

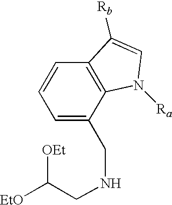

with stereoselectivity in the presence of Cbz-Tyrosine-OtBu and 2-(1-allyl-4-benzylsemicarbazido)acetic acid to provide

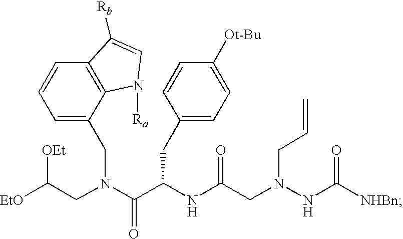

(d) cyclizing

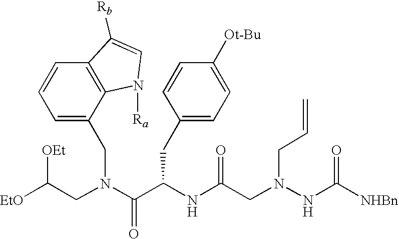

in the presence of formic acid to provide

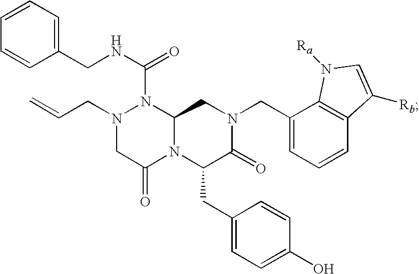

and (e) converting

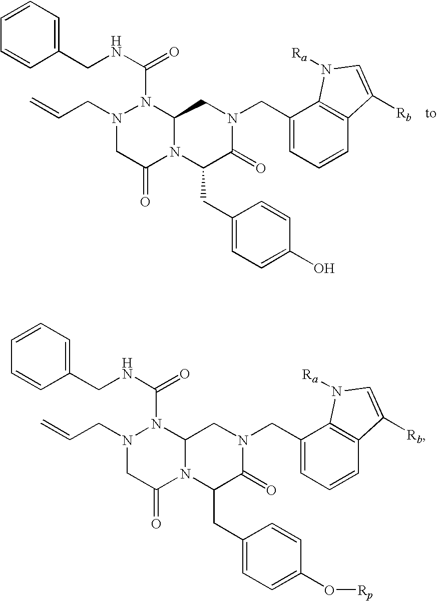

wherein Rₚ is —PO₃H₂, —HPO₃⁻Na⁺, —PO₃²⁻Na₂⁺, —PO₃²⁻K₂⁺, —PO₃²⁻Mg²⁺, —PO₃²⁻Ca²⁺. In certain embodiments, Rₐ is methyl, Rb is —C(═O)Rₑ, and Rₑ is methyl or cyclopropyl.

### BRIEF DESCRIPTION OF THE SEVERAL VIEWS OF THE DRAWINGS

FIG. 1 is a graph showing a correlation between the changes in pH and the potential conducted during the final step of the method for manufacturing the compound, in which 0.5 N NaOH is added dropwise to 4-(((6S,9aS)-1-(benzylcarbamoyl)-8-((3-acetyl-1-methyl-1H-indol-7-yl)methyl)-2-allyl-octahydro-4,7-dioxo-1H-pyrazino[2,1-c][1,2,4]triazin-6-yl)methyl)phenyl dihydrogen phosphate (Compound P2). In this graph, the horizontal axis represents the added amounts of NaOH. The first and second points of inflection correspond to the start of the production of monosodium and disodium, respectively.

### DETAILED DESCRIPTION

The novel reverse turn mimetics according to the present disclosure are observed to effectively inhibit the in vitro growth of AML cancer cells. Also, they are observed in testing of mice models of acute myeloid leukemia to effectively inhibit the growth of tumors.

Without wishing to be bound by theory, it is thought that as the leaving group (Rₚ), also referred to as the prodrug-functional group, is separated, the compounds of Chemical Formula I turn into active forms. However, these active forms are difficult to prepare into an aqueous solution due to their poor solubility in water. In the prodrug forms, the compounds of Chemical Formula I in accordance with the present disclosure are of high solubility and of high stability and are easy to be prepared as a preparation for injection.

Animal tests showed that the compounds of the present disclosure have excellent pharmaceutical efficacy. This seems to be attributable to the fast conversion of the compounds into their active forms just after intravenous injection, which results in a surge of an initial drug concentration. In this manner, the speed with which the prodrug compounds turn into active forms has influence on the medicinal efficacy thereof, so that it is important to choose prodrug-functional groups which allow optimal effects.

In a preferred embodiment, the prodrug functional groups are in the form of phosphate because the phosphate prodrugs are converted faster in vivo into active forms than the other prodrugs having other functional groups.

When the prodrug-functional groups are in the form of sodium salts, they are easy to prepare and have high solubility in water. In addition, they are highly stable during storage at room temperature.

Usually, a suitable injection composition is known to range in pH from 4 to 9, and preferably has a pH that is close to that of human blood, 7.4. A composition which is strongly acidic or strongly basic is not preferred as a composition for injection. In the case of a phosphate functional group, the final prodrugs of the present disclosure may be in the form of monosodium or disodium phosphate depending on the amount of sodium hydroxide. These compounds are advantageous for manufacturing a composition having pH values suitable for injection.

Further, the manufacturing method according to the present disclosure allows the production of not only compounds of Chemical Formula I, but also reverse turn mimetics thereof on an industrial scale.

Thus, one embodiment provides novel reverse turn mimetics, represented by the following Chemical Formula I, which are useful as therapeutic agents for cancer, in particular for acute myeloid leukemia.

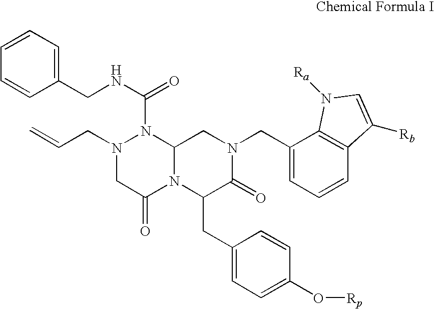

wherein

Rₚ may be any of the conventional functional groups which are available in prodrugs. Examples of the functional groups include phosphate, carboxy, and C₁-C₆ alkyamino, and acylamino, such as —PO₃H₂, —HPO₃⁻Na⁺, —PO₃²⁻Na₂⁺, —PO₃²⁻K₂⁺, —PO₃²⁻Mg²⁺, —PO₃²⁻Ca²⁺,

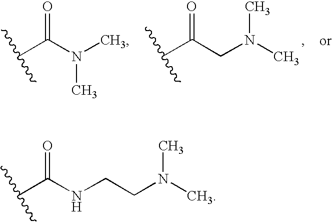

Preferably, Rₚ is a phosphate functional group

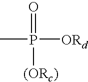

wherein R_{c} and R_{d} are same or different and independently H, Na, Mg, Ca or K. Preferably, both R_{c} and R_{d} are H or Na, or one of them is Na while the other is H.

Rₚ may also be —H, the resulting chemical structure is an active form of the corresponding prodrug as the prodrug functional group is removed.

Rₐ is an alkyl group, an alkenyl group, or an alkynyl group; preferably a C₁-C₆ alkyl group, a C₂-C₆ alkenyl, or a C₂-C₆ alkynyl group; and more preferably a C₁-C₆ alkyl group.

R_{b} is an aryl group, a substituted aryl group, or —C(═O)Rₑ wherein Rₑ is a C₁-C₆ alkyl group, a C₂-C₆ alkenyl group, or a C₂-C₆ alkynyl, and the substituted aryl group is a acyl-substituted aryl group and preferably acyl-substituted phenyl.

The compounds in prodrug from turn into active forms in the body. When the prodrugs have the phosphate functional group as a leaving group, the —PO₃RₚR_{d} group is rapidly cleaved by phosphatase and the prodrugs change into the active forms thereof. At this time, Rₚ is changed into —H (a chemical structure in active form as the prodrug functional group has left from the structure).

As used herein, the term “alkyl” or “alkyl group” is intended to include linear, branched or cyclic hydrocarbon radical comprising carbon and hydrogen atoms, wherein the carbon atoms are linked together by single bonds. In some embodiment, alkyl contains up to 20 carbons. In preferred embodiments, an alkyl may comprise one to six carbon atoms and be represented by “C₁-C₆ alkyl.” An alkyl is attached to the rest of the molecule by a single bond. Examples of alkyls include, without limitation, methyl, ethyl, n-propyl, 1-methylethyl (iso-propyl), n-butyl, n-pentyl, n-hexyl, 1,1-dimethylethyl (t-butyl), 2,2-dimethylpropyl (neo-pentyl), 3-methylhexyl, 2-methylhexyl, and the like. An alkyl may also be a monocyclic or bicyclic hydrocarbon ring radical, which may include fused or bridged ring systems. A cyclic alkyl is also referred to as “cycloalkyl.” In certain embodiments, a cycloalkyl may comprise three to six carbon atoms and be represented by “C₃₋₆cycloalkyl.” Examples of monocyclic cycloalkyl radicals include, e.g., cyclopropyl, cyclobutyl, cyclopentyl, cyclohexyl, cycloheptyl, and cyclooctyl.

“Alkenyl” or “alkenyl group” refers to linear, branched or cyclic hydrocarbon radical comprising carbon and hydrogen atoms, wherein at least two carbon atoms are linked by a double bond. In some embodiment, alkyl contains up to 20 carbons. In preferred embodiments, an alkenyl may comprise two to six carbon atoms and be represented by “C₂-C₆ alkyl.” An alkenyl is attached to the rest of the molecule by a single or double bond. Examples of alkenyls include, without limitation, ethenyl, allyl, butenyl and the like.

“Alkynyl” or “alkynyl group” refers to linear, branched or cyclic hydrocarbon radical comprising carbon and hydrogen atoms, wherein at least two carbon atoms are linked by a triple bond. In some embodiment, alkyl contains up to 20 carbons. In preferred embodiments, an alkynyl may comprise two to six carbon atoms and be represented by “C₂-C₆ alkynyl.” An alkynyl is attached to the rest of the molecule by a single bond. Examples of alkynyls include, without limitation, ethynyl, 1-propynyl, or 2-propynyl and the like.

Unless stated otherwise specifically in the specification, the term “alkyl” is meant to include an alkyl having solely carbon and hydrogen atoms as well as “substituted alkyl,” which refers to an alkyl radical in which one or more hydrogen atoms are replaced by one or more substituents independently selected from: acyl, alkoxy, aryl, cyano, cycloalkyl, halo, hydroxyl, nitro, —OC(O)—R¹¹, —N(R¹¹)₂, —C(O)OR¹¹, —C(O)N(R¹¹)₂, —N(R¹¹)C(O)OR¹¹, —N(R¹¹)C(O)R¹¹, —N(R¹¹)S(O)ₜR¹¹ (where t is 1 or 2), —S(O)ₜOR¹¹ (where t is 1 or 2), —S(O)ₚR¹¹ (where p is 0, 1 or 2), and —S(O)ₜN(R¹¹)₂ (where t is 1 or 2) where each R¹¹ is independently hydrogen, alkyl, aryl, as defined herein. The terms “alkenyl” and “alkynyl” are likewise defined as including “substituted alkenyl” and “substituted alkynyl,” respectively.

“Alkoxy” refers to a radical represented by the formula alkyl-O—, wherein alkyl is as defined herein. The alkyl portion can be further substituted by one or more halogen. An alkoxy may also be represented by the number of the carbons in the alkyl group, for example, C₁₋₆alkoxy or C₁₋₃alkoxy.

“Acyl” refers to a radical represented by the formula R¹²C(═O)—, wherein R¹² is alkyl or aryl as defined herein. The alkyl or aryl can be optionally substituted with the substituents as described for an alkyl or an aryl group, respectively. Exemplary acyl groups include, without limitation, methylacyl (i.e., acetyl), phenylacyl, cyclopropylacyl, and the like.

“Aryl” refers to a radical derived from an aromatic monocyclic or bicyclic ring system by removing a hydrogen atom from a ring carbon atom. The aromatic monocyclic or bicyclic hydrocarbon ring system comprises six to twelve carbon atoms (i.e., C₆₋₁₂ aryl), wherein at least one of the rings in the ring system is fully unsaturated, i.e., it contains a cyclic, delocalized (4n+2)π-electron system in accordance with the Hückel theory. Examples of aryl radicals include, but are not limited to, phenyl and naphthyl. Unless stated otherwise specifically in the specification, the term “aryl” is meant to include both aryl and “substituted aryl,” which refers to an aryl radical in which one or more hydrogen atoms are replaced by one or more substituents independently selected from: acyl, alkoxy, aryl, cyano, cycloalkyl, halo, hydroxyl, nitro, —OC(O)—R¹¹, —N(R¹¹)₂, —C(O)OR¹¹, —C(O)N(R¹¹)₂, —N(R¹¹)C(O)OR¹¹, —N(R¹¹)C (O)R¹¹, —N(R¹¹)S(O)ₜR¹¹ (where t is 1 or 2), —S(O)ₜOR¹¹ (where t is 1 or 2), —S(O)ₚR¹¹ (where p is 0, 1 or 2), and —S(O)ₜN(R¹¹)₂ (where t is 1 or 2) where each R¹¹ is independently hydrogen, alkyl, aryl, as defined herein.

“Halo” refers to fluoro, chloro, bromo and iodo.

The active form of the compounds is not suitable for I.V. injection due to the low solubility thereof in an aqueous medium (e.g., saline or water). The prodrug forms described herein are suitable for I.V. injection due to their improved solubility in the aqueous medium. In a preferred embodiment, a phosphate prodrug is used; and when one or two Na atoms were introduced at the phosphate moiety, the solubility is further improved. To introduce Na atoms, sodium hydroxide is added (e.g., dropwise) to the phosphate compound at a specific value of pH to perform substitution with one or two protons of the phosphate moiety with sodium ions.

Thus, a further embodiment provides a pharmaceutical composition comprising a compound of Chemical Formula (I) and a pharmaceutically acceptable excipient. The compounds or compositions of the present invention may be used in treating AML as described in detail below.

The pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In a preferred embodiment, the pharmaceutically acceptable excipient is suitable for use in I.V. administration, such as I.V. injection or infusion. Suitable carriers for I.V. administration include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi.

In other embodiments, oral compositions that generally include an inert diluent or an edible carrier are provided. Such compositions can be enclosed in gelatin capsules or compressed into tablets. For the purpose of oral therapeutic administration, compound described herein can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In accordance with another aspect, the present disclosure provides a method of treatment of diseases, particularly cancer, more particularly acute myeloid leukemia (AML) comprising administering to a cancer patient (e.g., a patient with AML) an effective amount of a compound of Chemical Formula (I) or a pharmaceutical composition comprising the same. Example 23 provides below demonstrates that exemplary compounds of the present disclosure are effective in treating AML in an animal model.

“Treatment” or “treating” refers to a treatment by which at least one symptom of disease in an individual receiving treatment improves, worsening of a progressive disease in an individual is delayed, or the onset of additional associated diseases is delayed or prevented.

An “effective amount” of a compound refers to that amount of the compound sufficient to result in amelioration of one or more symptoms of the disease being treated in a statistically significant manner.

Examples of the compounds of Chemical Formula (I) are given in Table 1 below. Because four compounds in Table 1 are different only in the Rₚ moiety which is H or phosphate functional group have the same NMR data, it is commonly given thereto in Table 1 (Rₚ was not observed in ¹H NMR spectra because it was substituted with deuterium).

TABLE 1

| No. | Cpd. | M.W. | NMR |
|---|---|---|---|
| 1 | 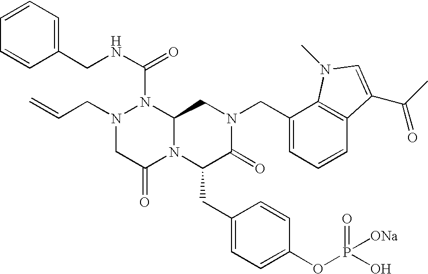 | 736.69 | ¹H NMR (500 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 5.87 (5, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), |
| 2 | 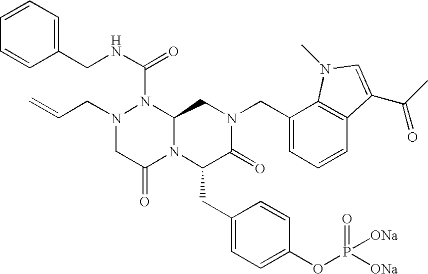 | 758.67 | 4.29 (dd, J = 9.0, 3.6 Hz, 1H), 4.02 (s, 3H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 2.51 (s, 3H) |
| 3 | 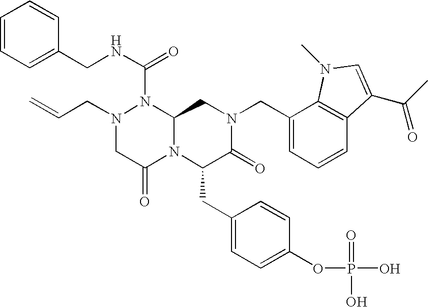 | 714.70 | |
| 4 | 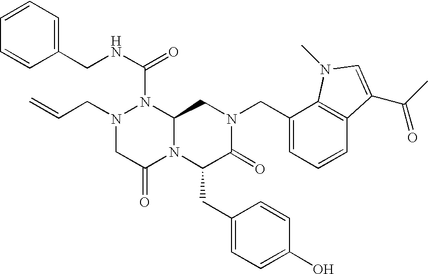 | 634.72 | |
| 5 | 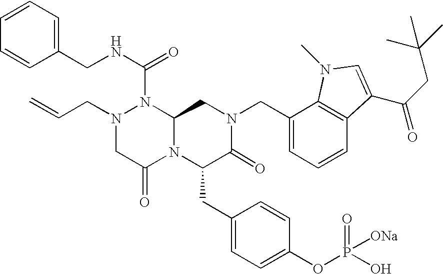 | 792.79 | ¹H NMR (D₂O, 300 MHz) δ 7.27 (d, 2H, J = 8.4 Hz), 7.175 (d, 1H, J = 7.2 Hz), 6.37~6.31 (m, 3H), 6.214 (d, 2H), 6.14~6.07 (m, 4H), 4.51~4.46 (dd, 2H), J = 10.8 Hz), 4.31~4.04 (dd, 2H, J = 14.7 Hz), 3.39~3.34 (d, 1H, J = 8.4 Hz), 3.34~2.97 (dd, 2H, J = 15.3, 15.3 Hz), 4.33 (dd, 1H, J = 15.3, 6.3 Hz), 2.97 (s, 3H), 2.75 (d, |
| 6 | 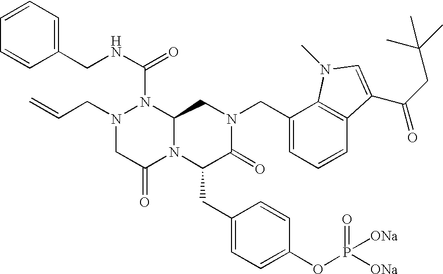 | 814.77 | 1H), 2.49~2.05 (dd, 2H, J = 15.3 Hz), 1.19 (s, 9H) |
| 7 | 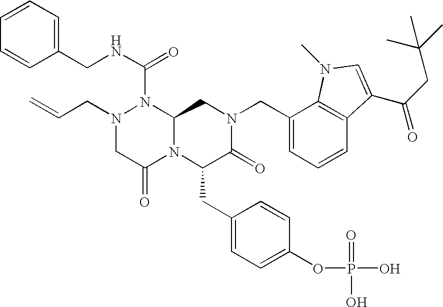 | 770.81 | |
| 8 | 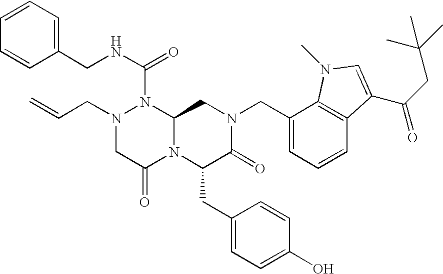 | 690.83 | |
| 9 | 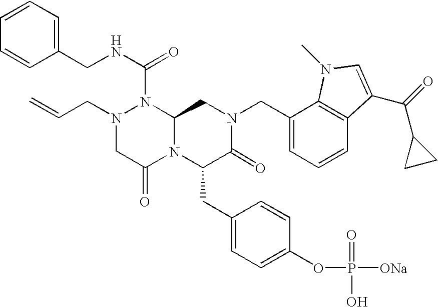 | 762.72 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), 4.29 (dd, J = 9.0, 3.6 Hz, 1H), 4.02 (s, |
| 10 | 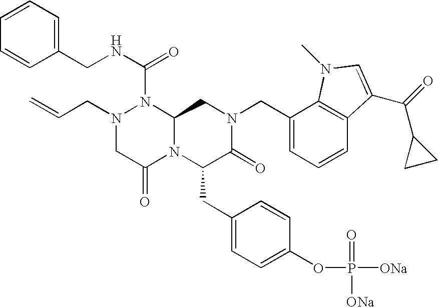 | 784.71 | 3H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 1.28 (m, 1H), 0.63 (m, 2H), 0.38 (m, 2H) |
| 11 | 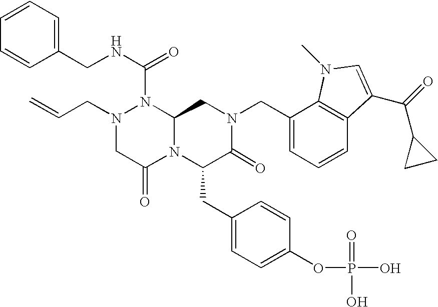 | 740.74 | |
| 12 | 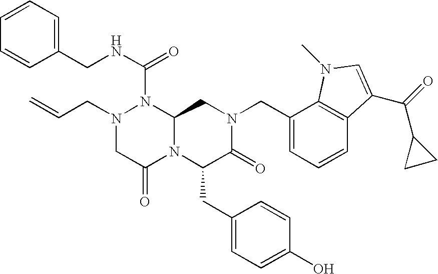 | 660.76 | |
| 13 | 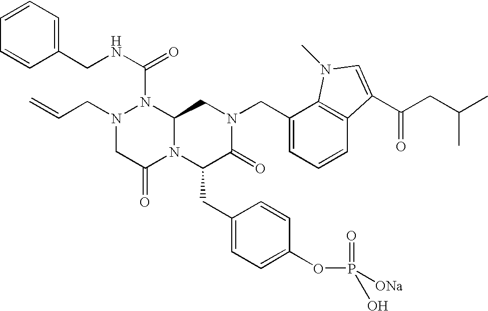 | 778.77 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), 4.29 (dd, J = 9.0, 3.6 |
| 14 | 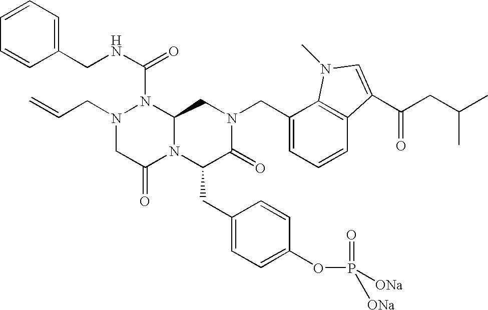 | 800.75 | Hz, 1H), 4.02 (s, 3H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 2.51 (d, J = 5.0 Hz, 2H). 2.06 (m, 1H), 1.01 (d, J = 5.2 Hz, 6H) |
| 15 | 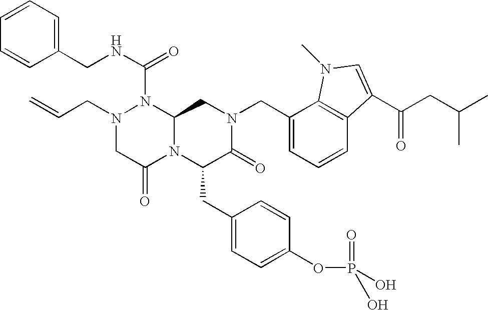 | 756.78 | |
| 16 | 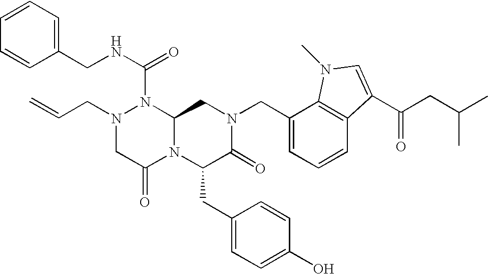 | 676.80 | |
| 17 | 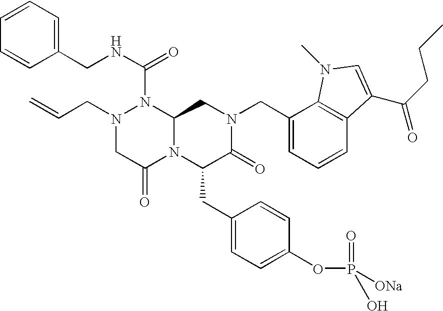 | 764.74 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), 4.29 (dd, J = 9.0, 3.6 |
| 18 | 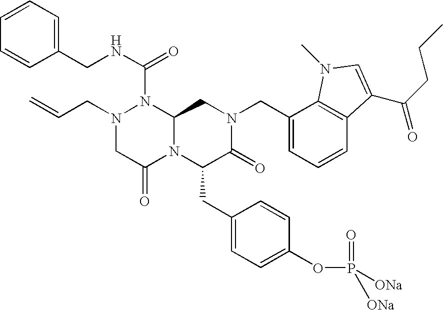 | 786.72 | Hz, 1H), 4.02 (s, 3H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 2.51 (d, J = 5.0 Hz, 2H). 1.66 (m, 2H), 0.98 (t, J = 4.2 Hz, 3H) |
| 19 | 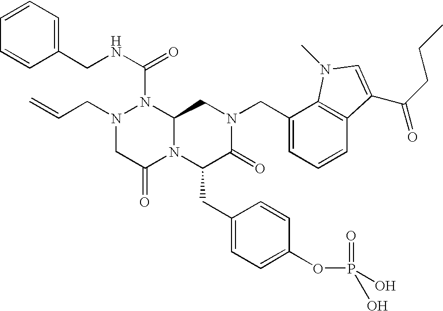 | | |
| 20 | 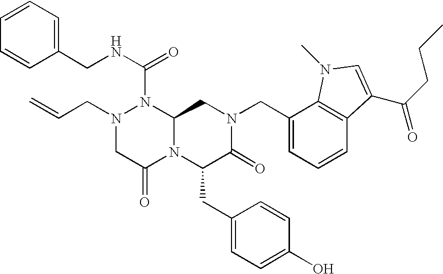 | 662.78 | |
| 21 | 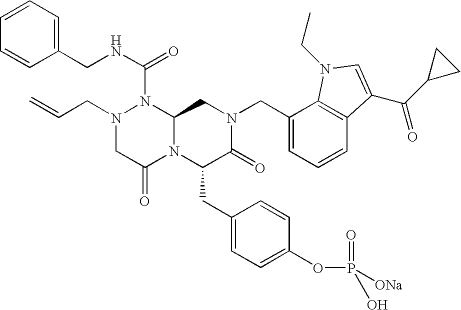 | 776.75 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), 4.29 (dd, J = 9.0, 3.6 |
| 22 | 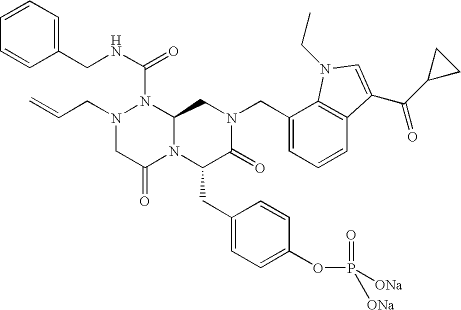 | 798.73 | Hz, 1H), 4.02 (q, J = 4.8 Hz, 2H), 3.43 (d, J = 7.2 Hz, 1H), 3.38- 3.33 (m, 3H), 3.27 (d, J = 7.2 Hz, 1H), 3.29-3.24 (m, 1H), 3.18 (dd, J = 7.2, 2.4 Hz, 1H), 1.51 (t, J = 5 Hz, 3H), 1.28 (m, 1H), 0.63 (m, 2H), 0.38 (m, 2H) |
| 23 | 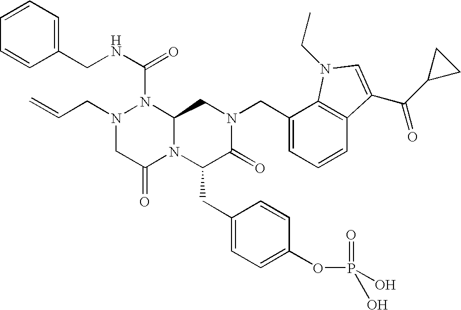 | 754.77 | |
| 24 | 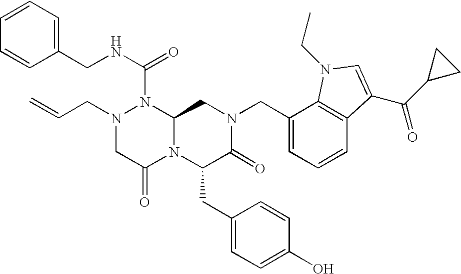 | 674.79 | |
| 25 | 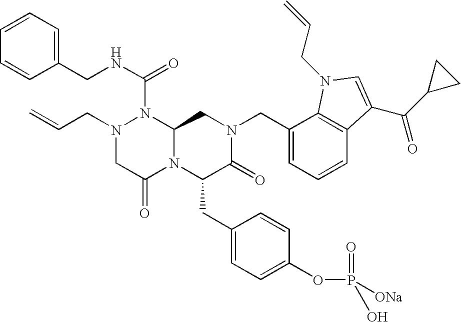 | 788.76 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.83 (m, 1H), 5.55-5.44 (m, 3H), 5.34 (t, J = 4.6 Hz, 1H), 5.17 (m, 2H), 5.03 (d, J = 6.3 Hz, 1H), 4.87 (d, J = 9.0 Hz, 1H), 4.79 9d, J = 7.5 Hz, 1H), 4.42 (dd, J = 9.0, 3.6 Hz, 1H), 4.29 |
| 26 | 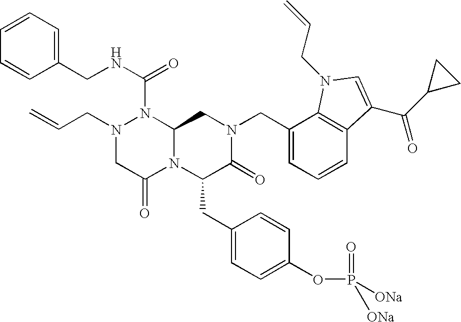 | 810.74 | (dd, J = 9.0, 3.6 Hz, 1H), 4.02 (m, 2H), 3.43 (d, J = 7.2 Hz, 1H), 3.38-3.33 (m, 3H), 3.27 9d, J = 7.2 Hz, 1H), 3.29-3.24 (m, 1H), 3.18 (dd, J = 7.2, 2.4 Hz, 1H), 1.28 (m, 1H), 0.63 (m, 2H), 0.38 (m, 2H) |
| 27 | 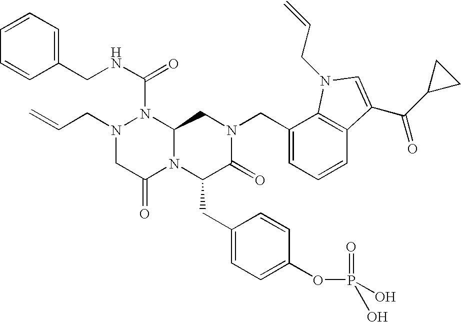 | 766.78 | |
| 28 | 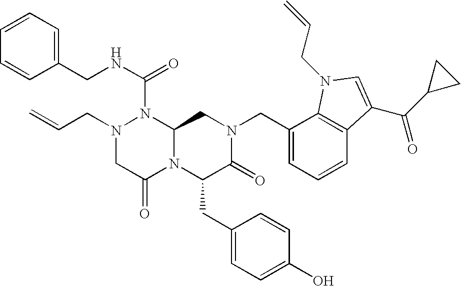 | 686.80 | |
| 29 | 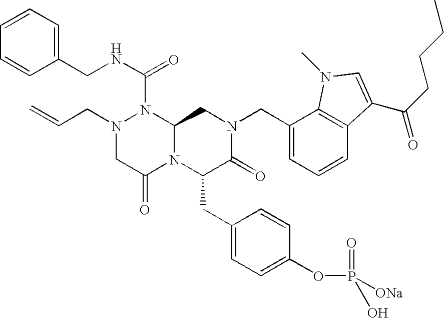 | 778.77 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), 4.29 (dd, J = 9.0, 3.6 Hz, 1H), 4.02 (s, |
| 30 | 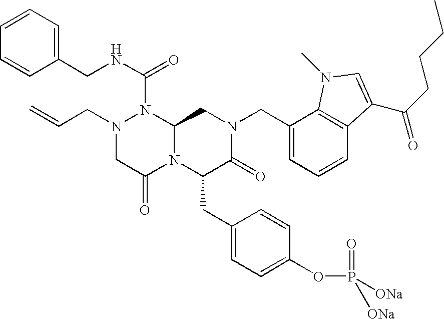 | 800.75 | 3H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 2.51 (d, J = 5.0 Hz, 2H). 1.62 (m, 2H), 1.33 (m, 2H), 0.96 (t, J = 4.0 Hz, 3H) |
| 31 | 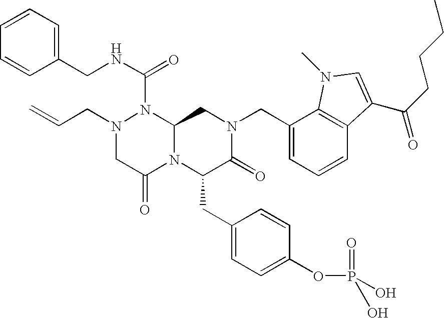 | 756.78 | |
| 32 | 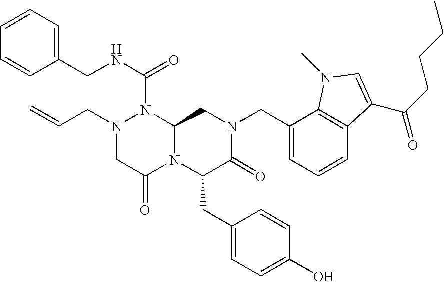 | 676.80 | |
| 33 | 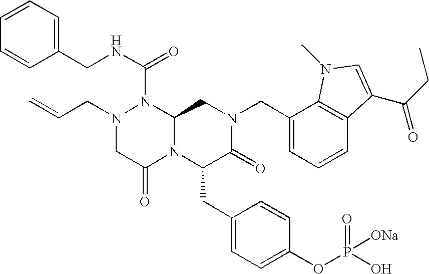 | 750.71 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, |
| 34 | 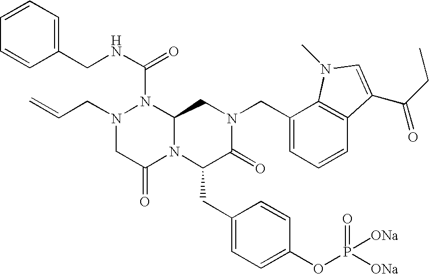 | 772.69 | J = 9.0, 3.6 Hz, 1H), 4.29 (dd, J = 9.0, 3.6 Hz, 1H), 4.02 (s, 3H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 2.44 (q, J = 4.1 Hz, 2H). 1.18 (t, J = 4.1 Hz, 3H) |
| 35 | 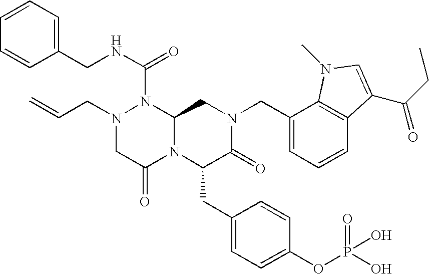 | 728.73 | |
| 36 | 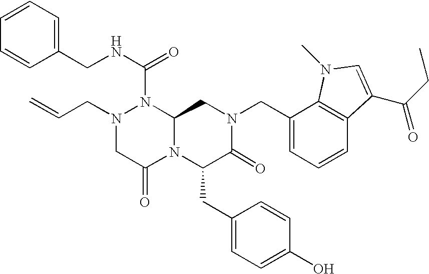 | 648.75 | |
| 37 | 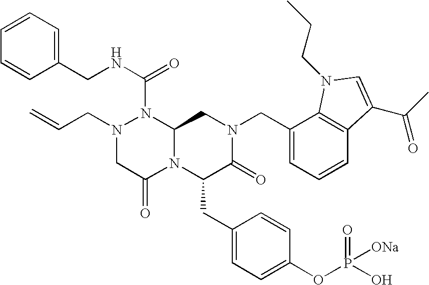 | 764.74 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), |
| 38 | 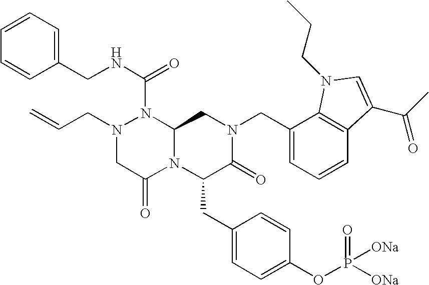 | 786.72 | 4.29 (dd, J = 9.0, 3.6 Hz, 1H), 3.85 (m, 2H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1H), 2.51 (s, 3H). 1.81 (m, 2H), 0.96 (t, J = 4.3 Hz, 3H) |
| 39 | 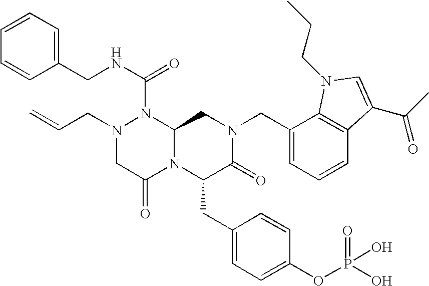 | 742.76 | |
| 40 | 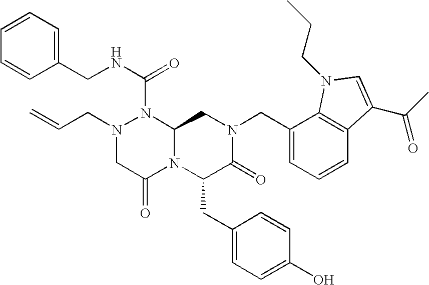 | 662.78 | |
| 41 | 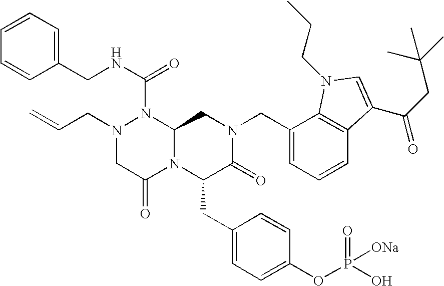 | 820.85 | ¹H NMR (D2O, 300 MHz) δ 7.27 (d, 2H, J = 8.4 Hz), 7.175 (d, 1H, J = 7.2 Hz), 6.37~6.31 (m, 3H), 6.214 (d, 2H), 6.14~6.07 (m, 4H), 4.51~4.46 (dd, 2H, J = 10.8 Hz), 4.31~4.04 (dd, 2H, J = 14.7 Hz), 3.39~3.34 (d, 1H, J = 8.4 Hz), 3.34~2.97 (dd, 2H, J = 15.3, 15.3 Hz), 4.33 (dd, 1H, J = 15.3, 6.3 Hz), 2.97 (m, 2H), 2.75 (d, 1H), 2.49~2.05 |
| 42 | 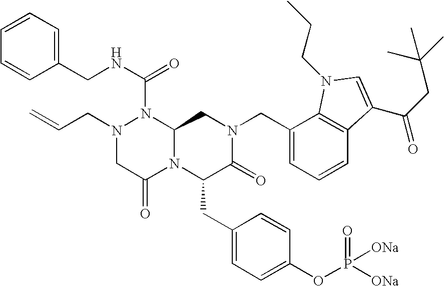 | 842.83 | (dd, 2H, J = 15.3 Hz), 1.92 (m, 2H), 1.20 (s, 9H), 1.01 (t, J = 4.3 Hz, 3H) |
| 43 | 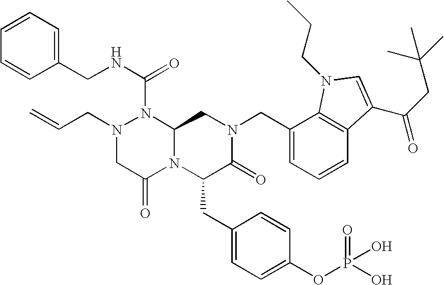 | 798.86 | |
| 44 | 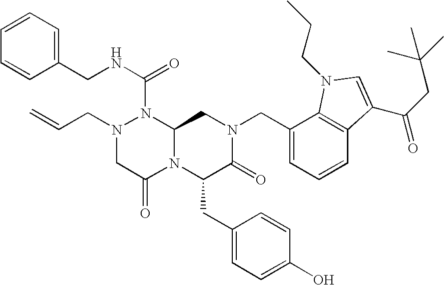 | 718.88 | |
| 45 | 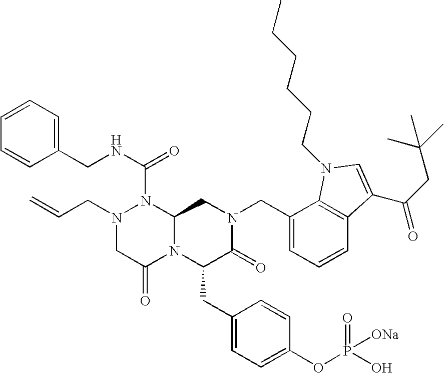 | 862.93 | ¹H NMR (D₂O, 300 MHz) δ 7.27 (d, 2H, J = 8.4 Hz), 7.175 (d, 1H, J = 7.2 Hz), 6.37~6.31 (m, 3H), 6.214 (d, 2H), 6.14~6.07 (m, 4H), 4.51~4.46 (dd, 2H, J = 10.8 Hz), 4.31~4.04 (dd, 2H, J = 14.7 Hz), 3.39~3.34 (d, 1H, J = 8.4 Hz), 3.34~2.97 (dd, 2H, J = 15.3, 15.3 Hz), 4.33 (dd, 1H, J = 15.3, 6.3 Hz), 2.97 (m, 2H), 2.75 (d, 1H), 2.49~2.05 (dd, 2H, J = 15.3 Hz), 1.77~1.30 (m, 8H), 1.20 (s, 9H) 0.98 (t, J = 5.0 Hz, 3H) |
| 46 | 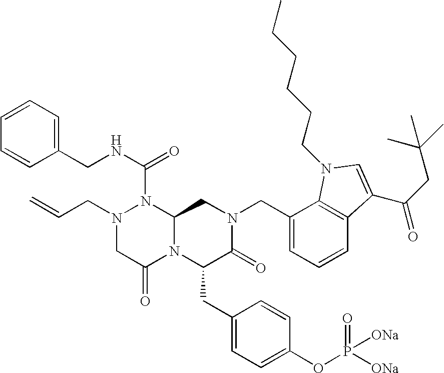 | 884.91 | |
| 47 | 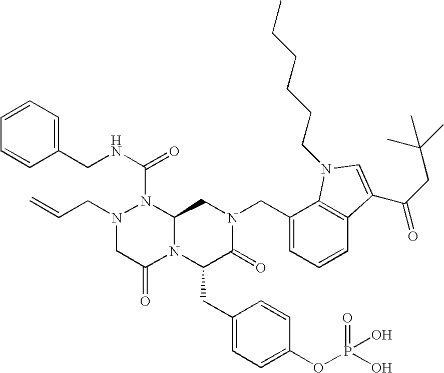 | 840.94 | |
| 48 | 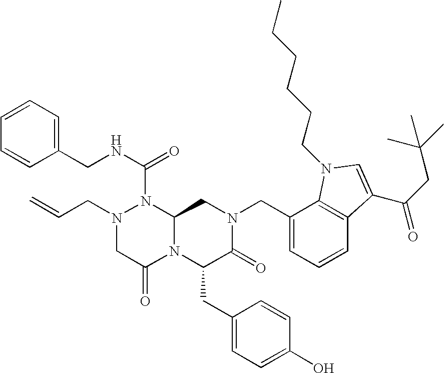 | 760.96 | |
| 49 | 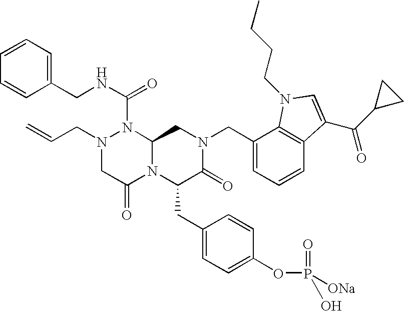 | 804.80 | ¹H NMR (300 MHz, CDCl₃) δ 8.43 (d, J = 4.8 Hz, 1H), 7.63 (s, 1H), 7.38-7.35 (m, 2H), 7.31-7.30 (m, 1H), 7.29-7.21 (m, 2H), 7.00 (d, J = 4.8 Hz, 2H), 6.97 (d, J = 4.8 Hz, 1H), 6.69-6.65 (m, 3H), 5.87 (s, 1H), 5.55- 5.44 (m, 3H), 5.34 (t, J = 4.6 Hz, 1H), 5.03 (d, J = 6.3 Hz, 1H), 4.87 (d, J = 9.0 Hz, 1H), 4.79 (d, J = 7.5 Hz, 1H), 4.42 (dd, J = 9.0, 3.6 Hz, 1H), 4.29 (dd, J = 9.0, 3.6 |
| 50 | 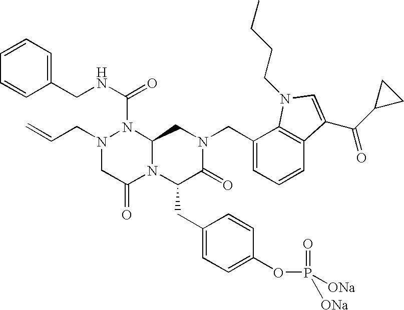 | 826.78 | Hz, 1H), 4.02 (m, 2H), 3.43 (d, J = 7.2 Hz, 1H), 3.38-3.33 (m, 3H), 3.27 (d, J = 7.2 Hz, 1H), 3.29- 3.24 (m, 1H), 3.18 (dd, J = 7.2, 2.4 Hz, 1 H), 1.77 (m, 2H), 1.88 (m, 2H), 1.28 (m, 1H), 0.98 (t, J = 4.8 Hz, 3H), 0.63 (m, 2H), 0.38 (m, 2H) |
| 51 | 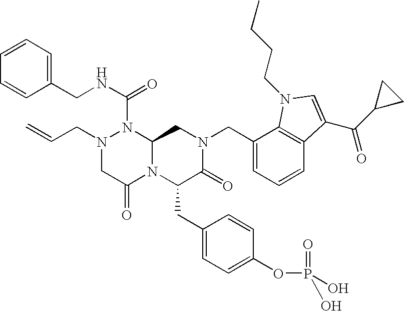 | 782.82 | |
| 52 | 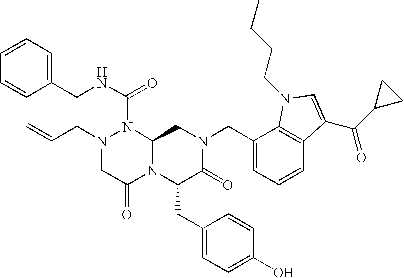 | 702.84 | |
| 53 | 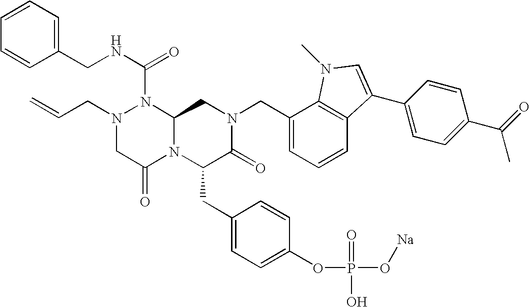 | 812.78 | ¹H NMR (CDCl₃, 300 MHz) δ 8.05 (d, 2H, J = 8.4 Hz), 7.91 (d, 1H, J = 7.2 Hz), 7.71 (d, 2H, J = 8.4 Hz), 7.40~7.20 (m, 4H), 7.16 (t, 1H, J = 7.2Hz), 7.05 (d, 2H, J = 8.4 Hz), 6.96 (d, 1 H, J = 6.9 Hz), 6.69 (d, 2H, J = 8.4 Hz), 6.68(m, 1H), 5.58~5.44 (m, 3H), 5.37 (t, 1 H, J = 5.7 Hz), 5.03 (d, 1H, J = 10.8 Hz), 4.97 (d, 1H, J = 14.7 Hz), 4.81 (d, 1H, J = 17.1 Hz), 4.47 (dd, 1H, J = 15.3, 6.3 Hz), 4.33 (dd, 1 H, |
| 54 | 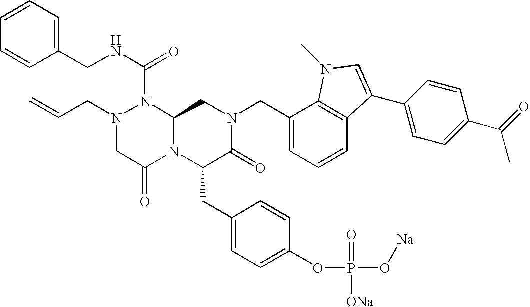 | 834.76 | J = 15.3, 6.3 Hz), 4.33 (s, 3H), 3.47~3.24 (m, 8H), 2.64 (s, 3H) |

Methods known in the art may be used to determine the effectiveness of a compound provided here in treating cancer, such as AML. For example, the method described in Example 23 may be used for assessing the anticancer activity of a given compound. Additional exemplary methods for assessing the activity of a compound in treating AML include those described in Bishop et al., Blood 87: 1710-7, 1996; Bishop, Semin Oncol 24:57-69, 1997; and Estey, Oncology 16: 343-52, 2002.

The compounds of the present disclosure may be administered to a patient in need thereof via various routes, such as orally, topically, transdermally, or parenterally. In one embodiment, the compounds or compositions thereof are administered parenterally. The term “parenteral,” as used herein, includes subcutaneous injections, intravenous, intramuscular, intracisternal injections, and intravenous infusions. In preferred embodiments, the compounds or compositions are administered via injection, such as intravenous injections.

Toxicity and therapeutic efficacy of compounds of the present disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. In vitro cardiotoxicity of the compounds may be determined according to the method described in Example 24 below. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The effective dose depends on the type of disease, the composition used, the route of administration, the type of subject being treated, the physical characteristics of the specific subject under consideration for treatment, concurrent medication, and other factors that those skilled in the medical arts will recognize. For example, for treating AML, a compound of the present disclosure may be administered via I.V. injection or infusion at an amount between 0.5 mg/kg and 500 mg/kg (e.g., 0.5 to 10 mg/kg, 10 to 100 mg/kg, about 100 to 500 mg/kg body weight), which can be administered as a single dose, daily, weekly, monthly, or at any appropriate interval. In certain embodiments, the disclosed compounds may be used in treating AML in a manner similar to that used for Ara-C.

In accordance with a further aspect thereof, the present disclosure provides a method for manufacturing the reverse turn mimetics of the present disclosure on a mass scale. The method comprises the following sequential steps:

introducing an acyl group into indole-7-carbaldehyde, preferably through Friedel-Crafts acylation to provide 3-acyl-indole-7-carbaldehyde;

introducing an alkyl group and an aminoacetal group to 3-acyl-indole-7-carbaldehyde to provide a 1-alkyl-3-acyl-indole derivative;

amidating the 1-alkyl-3-acyl-indole derivative with stereoselectivity with Cbz-Tyr(OtBu) (i.e., (S)-2-(benzyloxycarbonylamino)-3-(4-tert-butoxyphenyl)propanoic acid) and 2-(1-allyl-4-benzylsemicarbazido)acetic acid to provide a reaction intermediate;

cyclizing the reaction intermediate in the presence of formic acid to provide a cyclic intermediate; and

phosphorylating the cyclic intermediate.

In the above method, 2-(1-allyl-4-benzylsemicarbazido)acetic acid may be prepared by the following sequential steps:

adding TEA(triethylamine) to an ethylhydrazinoacetate solution to form a reaction solution;

adding allyl bromide (e.g., dropwise) to the reaction solution; and

adding benzylisocyanate (e.g., dropwise).

Representative compounds of the disclosure can be prepared as illustrated in the following Reaction Scheme.

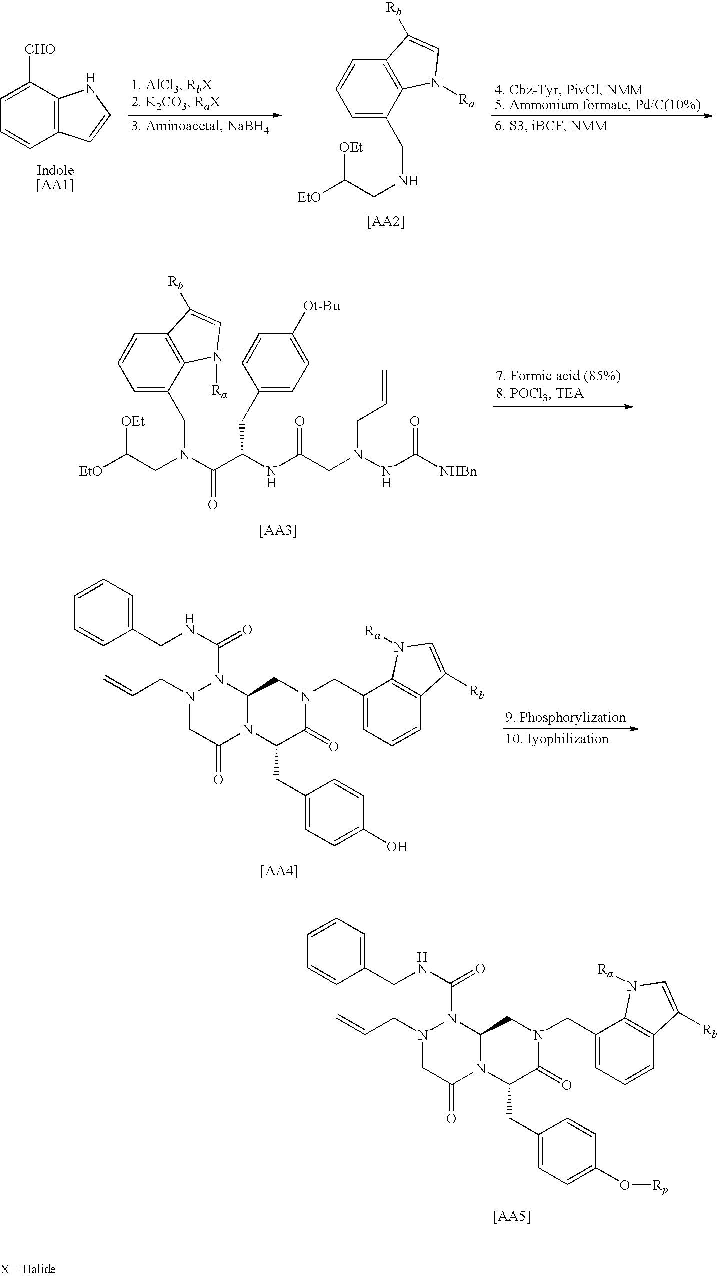

In certain embodiments, Rₐ is methyl, Rb is —C(═O)Rₑ, and Rₑ is methyl or cyclopropyl.

As seen herein, the reaction scheme is directed to novel reverse turn mimics, represented by Chemical Formula I.

The compounds according to the present disclosure are based on a framework of pyrazino-triazinone, with four different functional groups attached thereto. Due to the two chiral centers thereof, the compounds must be synthesized stereoselectively.

An acyl group is introduced into the indole-7-carbaldehyde of AA1 through Friedel-Crafts acylation, followed by the introduction of alkyl and aminoacetal groups.

After the reaction of AA2 with the chiral compound (Cbz-Tyrosine-OtBu), the resulting intermediate is subjected to stereoselective amidation with PivCl (Pivaloylchloride) and iBCF (isobutylchloroformate) to afford AA3. Thereafter, AA3 is cyclized with formic acid to obtain AA4, followed by phosphorylization, introduction of salt (addition of Na to phosphate using 0.5N NaOH) and lyophilization to synthesize highly pure pyrazino-triazone compounds, AA5.

A better understanding of the present disclosure may be obtained through the following examples which are set forth to illustrate, but are not to be construed as limiting the present disclosure.

As demonstrated herein, the compounds of Chemical Formula I exhibit anticancer activity.

The manufacturing method of the present disclosure is illustrated in detail as follows.

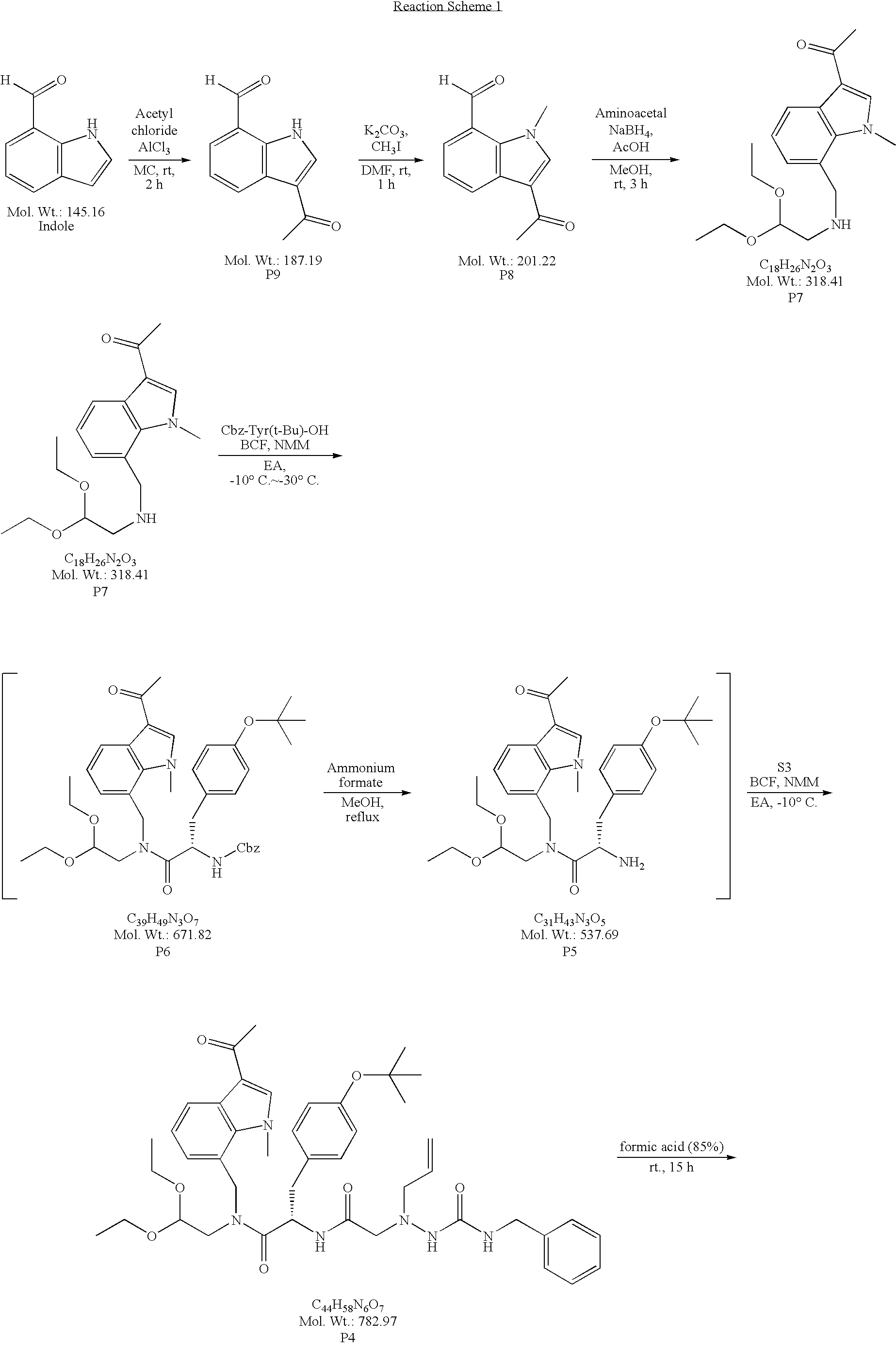

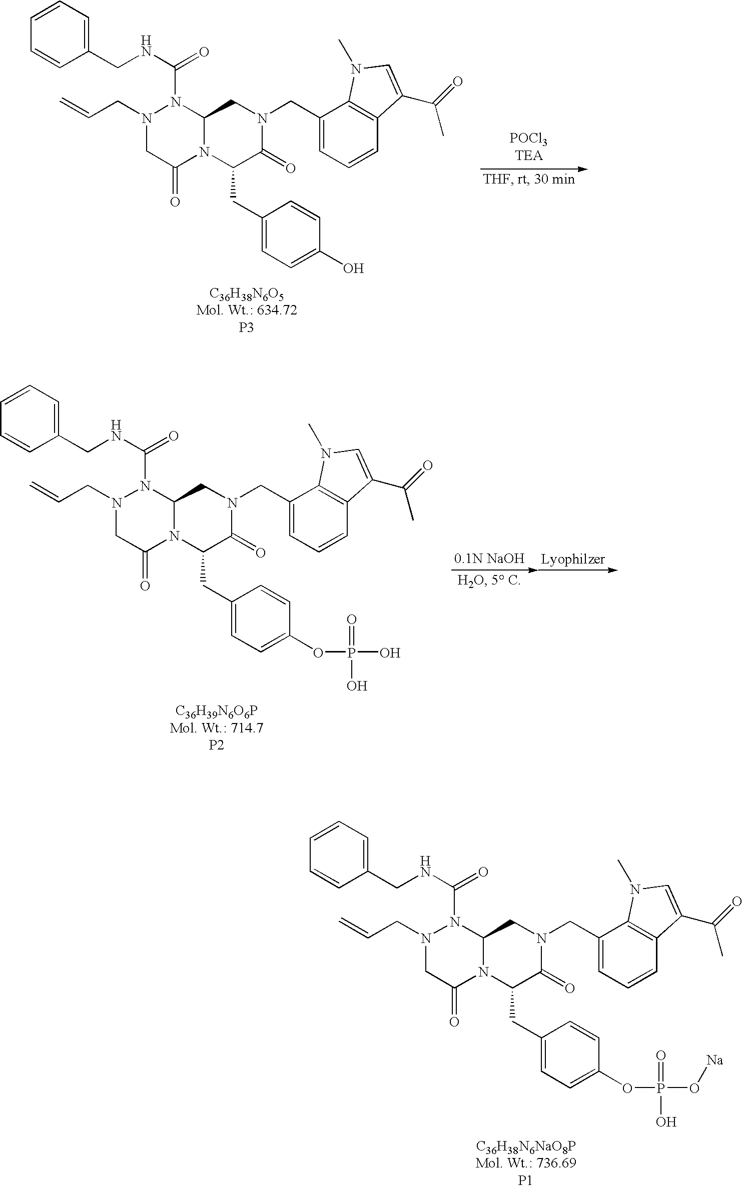

In Reaction Scheme 1, side chain S3 may be prepared as illustrated below.

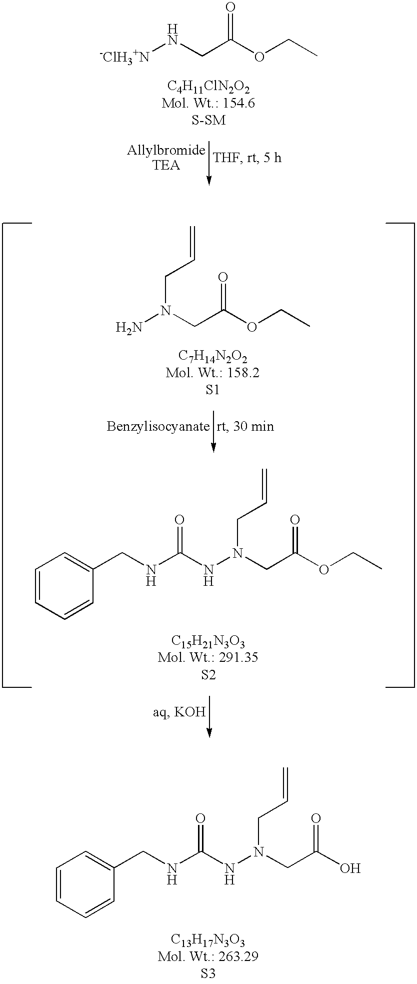

Below, each step of the manufacturing method illustrated in Reaction Scheme 1 will be described in detail in Examples 1 to 10.

### EXAMPLES

### Example 1

### Synthesis of S3

### 2-(1-allyl-4-benzylsemicarbazido)acetic acid

67 g of ethyl hydrazinoacetate was dissolved in 673 ml of THF (tetrahydrofuran) and mixed with 121 ml of TEA (triethylamine). To this reaction mixture was dropwise added 41 ml of allyl bromide over 20 min. This solution was stirred for 5 hrs and filtered. To the filtrate was dropwise added 53 ml of benzylisocyanate over 15 min, followed by stirring for 30 min at room temperature. Thereafter, a solution of 48 g of KOH (potassium hydroxide) in 673 ml of distilled water was dropwise added before stirring for 30 min. Layer separation was generated by adding 403 ml of MC (dichloromethane) and 269 ml of hexane and stirring. The aqueous solution was washed once with 201 ml of MC (dichloromethane). The aqueous solution was adjusted to a pH of 2˜3 by using 100 ml of conc. HCl. After being stirred for 30 min, the pH-adjusted solution was extracted with 1009 ml of MC (dichloromethane). The MC (dichloromethane) layer thus obtained was dehydrated with 269 g of Na₂SO₄, filtered, and then concentrated in a vacuum. The concentrate is crystallized with 134 ml of EA (ethylacetate) and 269 ml of hexane, followed by filtration. The solid thus obtained was slurried in 134 ml of EA (ethylacetate), filtered at 0° C. and dried in a vacuum to produce 40 g of S3 as a white solid (yield 35%).

¹H NMR (500 MHz, CDCl₃) δ 10.84 (bs, 1H), δ 7.90 (s, 1H), δ 7.4-7.3 (m, 5H), δ 6.42 (t, J=5.0 Hz, 1H), δ 5.85-5.72 (m, 1H), δ 5.28 (dd, J=28.5, 2.0 Hz, 1H), δ 5.19 (d, J=17 Hz, 1H), δ 4.47-4.42 (m, 2H), δ 3.70 (dd, J=40.0, 2.5 Hz, 1H).

### Example 2

### Synthesis of P9

### 3-acetyl-1H-indole-7-carbaldehyde

23.5 ml of AcCl(acetylchloride) was dropwise added to a solution of 55 g of AlCl₃ in 400 ml of MC (dichloromethane) with stirring. To this solution was dropwise added a solution of 40 g of the starting material (indole-7-carbaldehyde) in 400 ml of MC (dichloromethane). The temperature of the solution must be maintained at 0˜5° C. upon the addition and then allowed to increase to room temperature. The progress of the reaction was monitored using thin layer chromatography (TLC) and high performance liquid chromatography. After the reaction was completed, the solution was subjected to layer separation with water. The organic layer thus formed was dried over MgSO₄ (magnesium sulfate), filtered and then concentrated at 40° C. to give 41 g of P9 as concentrated residue (yield 80%).

### Example 3

### Synthesis of P8

### 3-acetyl-1-methyl-1H-indole-7-carbaldehyde

41 g of P9 was dissolved in 412 ml of DMF (dimethylformamide) and stirred. After the solution was cooled to 10° C., 91 g of K₂CO₃ (potassium carbonate) was added thereto, and 20 ml of MeI (methyliodide) was dropwise added. The resulting solution was allowed to increase in temperature to room temperature and was stirred for 4˜5 hrs. When the starting material was recognized as disappearing, K₂CO₃ was filtered off, followed by crystallization in hexane to give 35 g of P8 as a yellowish solid (yield 80%).

### Example 4

### Synthesis of P7

### 1-(7((2,2-diethoxyethylamino)methyl)-1-methyl-1H-indol-3-yl)ethanone

To a solution of 35 g of P8 in 354 ml of MeOH (methanol) was added 3.5 ml of AcOH (acetic acid). The solution was mixed with 33 ml of aminoacetaldehyde diethylacetal at room temperature and stirred for 3-4 hrs. After the solution was cooled to 10° C., 3.3 g of the reducing agent NaBH₄ (sodiumborohydride) was slowly added thereto. At this time, care had to be taken because of hydrogen gas generation and exothermal reaction. The solution was stirred at room temperature for 1 hr. When the reaction was completed, 354 ml of EA (ethylacetate) and 354 ml of distilled water were added so as to separate layers. The organic layer thus formed was dried over 141 g of MgSO₄ (magnesium sulfate) and crystallized in hexane to afford 85 g of P7 as a yellowish solid (yield 80%).

¹H NMR (500 MHz, CDCl₃), δ 8.36 (d, J=4.8 Hz, 1H), δ 7.61 (s, 1H), δ 7.17 (d, J=4.2 Hz, 1H), δ 7.10 (d, J=4.2 Hz, 1H), δ 4.58 (t, J=3.3, 1H), δ 4.21 (s, 3H), δ 4.07 (s, 3H), δ 3.68 (m, 2H), δ 3.51 (m, 2H), δ 2.82 (d, J=3.3 Hz, 2H), δ 2.48 (s, 3H), δ 1.19 (t, J=4.2 Hz, 6H).

### Example 5

### Synthesis of P6 benzyl(S)-1-(N-((3-acetyl-1-methyl-1H-indol-7-yl)methyl)-N-(2,2-diethoxyethyl)carbamoyl)-2-(4-tert-butoxyphenyl)ethylcarbamate

85 g of Cbz-Tyr(OtBu) was dissolved in 449 ml of EA (ethylacetate) with stirring. After the solution was cooled to 0˜5° C., 31 ml of NMM (N-methylmorpholine) and 19 ml of pivaloylchloroide were dropwise added thereto. The solution was stirred for 1˜2 hrs and then 44.9 g of P7 was added thereto at 0˜5° C. The solution was warmed to room temperature followed by stirring for 2-3 hrs. After termination of the reaction, distilled water was added to generate layer separation. The organic layer thus formed was washed with 898 ml of a 5% aqueous citric acid solution and 898 ml of a 5% aqueous NaHCO₃ solution and then dried over 179 g of MgSO₄ (magnesium sulfate) to be concentrated. 85 g of P6 was obtained as a residue (yield 90%).

### Example 6

### Synthesis of P5 (S)-3-(4-tert-butoxyphenyl)-N-((3-acetyl-1-methyl-1H-indol-7-yl)methyl)-2-amino-N-(2,2-diethoxyethyl)propanamide

To 85 g of P6 in 853 ml of MeOH was added 8.5 g of 10 wt % Pd/C. 16 g of ammonium formate was added and then refluxed for 2 hrs. After completion of the reaction, the solution was cooled to room temperature and Pd/C was filtered. The solution was concentrated before layer separation with 853 ml of EA (ethylacetate) and 1706 ml of distilled water. The organic layer thus formed was washed with 850 ml of a 5% aqueous citric acid solution and 850 ml of a 5% aqueous NaHCO₃ solution and concentrated to give 56 g of P5 (yield 90%).

### Example 7

### Synthesis of P4

40 g of side chain S3 was dissolved in 426 ml of EA (ethylacetate) and cooled to −10° C. To the solution were dropwise added 41 ml of NMM (N-methylmorpholine) and 20 ml of iBCF (iso-butylchloroformate) at the same temperature. The reaction mixture was stirred for 2˜3 hrs at −10° C. after which a solution of 56 g of P5 in 200 ml of EA (ethylacetate) was dropwise added thereto. The reaction mixture was warmed to room temperature and then stirred for 1˜2 hrs. When the reaction was terminated, EA (ethylacetate) and 850 ml of distilled water were added to separate layers. The organic layers thus formed was washed with 850 ml of a 5% aqueous citric acid solution and 850 ml of a 5% aqueous NaHCO₃ solution and dried over 340 g of MgSO₄ (magnesium sulfate) to the concentration. 81 g of P4 was obtained as a concentrated residue (yield 90%).

### Example 8

### Synthesis of P3

### (6S,9AS)-6-(4-hydroxybenzyl)-8-((3-acetyl-1-methyl-1H-indol-7-yl)methyl)-2-allyl-N-benzyl-hexahydro-4,7-dioxo-2H-pyrazino[2,1-C][1,2,4]triazine-1(6H)-carboxamide

81 g of P4 was dissolved in 383 ml of 85% formic acid and heated to 50° C. After being stirred for 1-2 hrs at the same temperature, the solution was cooled to room temperature and mixed with acetone. This solution was adjusted to a pH of 4.0˜4.2 by dropwise adding 5N NaOH, to form crude crystals. After cooling to 10˜15° C., the solid was filtered and completely dissolved in 767 ml of MeOH with warming. Slow cooling precipitated crystals which were filtered to afford P3 as a pinkish white crystal (40 g, yield 60%).

¹H NMR (500 MHz, CDCl₃) 8.43 (d, J=4.8 Hz, 1H), 7.63 (s, 1H), 7.38-7.35 (m, 2H), 7.31-7.30 (m, 1H), 7.29-7.21 (m, 2H), 7.00 (d, J=4.8 Hz, 2H), 6.97 (d, J=4.8 Hz, 1H), 6.69-6.65 (m, 3H), 5.87 (s, 1H), 5.55-5.44 (m, 3H), 5.34 (t, J=4.6 Hz, 1H), 5.03 (d, J=6.3 Hz, 1H), 4.87 (d, J=9.0 Hz, 1H), 4.79 (d, J=7.5 Hz, 1H), 4.42 (dd, J=9.0, 3.6 Hz, 1H), 4.29 (dd, J=9.0, 3.6 Hz, 1H), 4.02 (s, 3H), 3.43 (d, J=7.2 Hz, 1H), 3.38-3.33 (m, 3H), 3.27 (d, J=7.2 Hz, 1H), 3.29-3.24 (m, 1H), 3.18 (dd, J=7.2, 2.4 Hz, 1H), 2.51 (s, 3H).

### Example 9

### Synthesis of P2

### 4-(((6S,9AS)-1-(benzylcarbamoyl)-8-((3-acetyl-1-methyl-1H-indol-7-yl)methyl)-2-allyl-octahydro-4,7-dioxo-1H-pyrazino[2,1-c][1,2,4]triazin-6-yl)methyl)phenyl dihydrogen phosphate

40 g of P3 was dissolved in 217 ml of THF (tetrahydrofuran), cooled to 0˜5° C. and mixed with 25 ml of POCl₃. At the same temperature, 28 ml of TEA (triethylamine) was dropwise added. Stirring for 1 hr was followed by slow addition of 87 ml of distilled water. 348 ml of a sat. aqueous NaHCO₃ solution was added to the solution which was then stirred for 30 min. After the solution was subjected to layer separation by adding 217 ml of EA (ethylacetate), 217 ml of MC (methylenechloride) was added to the aqueous layer and then the pH was adjusted to 1˜3 with 14 ml of conc. HCl to separate layers. The organic layer thus formed was dehydrated with 174 g of Na₂SO₄ (sodium sulfate) and concentrated in a vacuum. The concentrate was crystallized in 130 ml of THF (tetrahydrofuran) and 435 ml of n-hexane, filtered, and vacuum dried to afford 40 g of P2 as a white solid (yield 90%).

¹H NMR (500 MHz, DMSO-d6) 8.27 (s, 1H), 8.16 (d, J=7.5 Hz, 1H), 7.85 (t, J=6.3 Hz, 1H), 7.34-7.29 (m, 3H), 7.22-7.01 (m, 9H), 6.79 (d, J=6.9 Hz, 1H), 5.84-5.75 (m, 1H), 5.52 (dd, J=8.1, 3.6 Hz, 1H), 5.38 (d, J=15.6 Hz, 1H), 5.17-5.13 (m, 1H), 5.09-5.03 (m, 2H), 4.90 (d, J=15.6 Hz, 1H), 4.22 (d, J=6.3 Hz, 2H), 4.06 (s, 3H), 3.76-3.68 (m, 1H), 3.61-3.55 (m, 2H), 3.33-3.27 (m, 4H), 3.07-3.02 (m, 2H), 2.41 (s, 3H).

### Example 10

### Synthesis of P1

### Sodium 4-(((6S,9AS)-1-(benzylcarbamoyl)-8-((3-acetyl-1-methyl-1H-indol-7-yl)methyl)-2-allyl-octahydro-4,7-dioxo-1H-pyrazino[2,1-C][1,2,4]triazin-6-yl)methyl)phenyl hydrogenphosphate

40 g of dried P2 was dissolved in 2000 ml of distilled water with stirring. The solution was cooled to 0˜5° C., followed by adjusting the pH thereof to 4.6˜4.8 (130˜110 mV) by slowly adding a 0.1 N aqueous NaOH solution, and then lyophilized to afford 40 g of P1 as a white solid (yield 95%).

¹H NMR (300 MHz, D2O) 7.86 (d, J=7.8 Hz, 1H), 7.60 (s, 1H), 7.07-6.93 (m, 10H), 6.56 (d, J=7.2 Hz, 1H), 5.39-5.32 (m, 2H), 5.09 (t, J=5.4 Hz, 1H), 4.95 (d, J=15.6 Hz, 1H), 4.70-4.53 (m, 2H), 4.14 (d, J=15.6 Hz, 1H), 3.97 (d, J=15.6 Hz, 1H), 3.57 (s, 3H), 3.56-3.49 (m, 1H), 3.30-2.81 (m, 6H), 2.84-2.81 (m, 1H), 2.18 (s, 3H).

Another preparation example for representative compounds is suggested below.

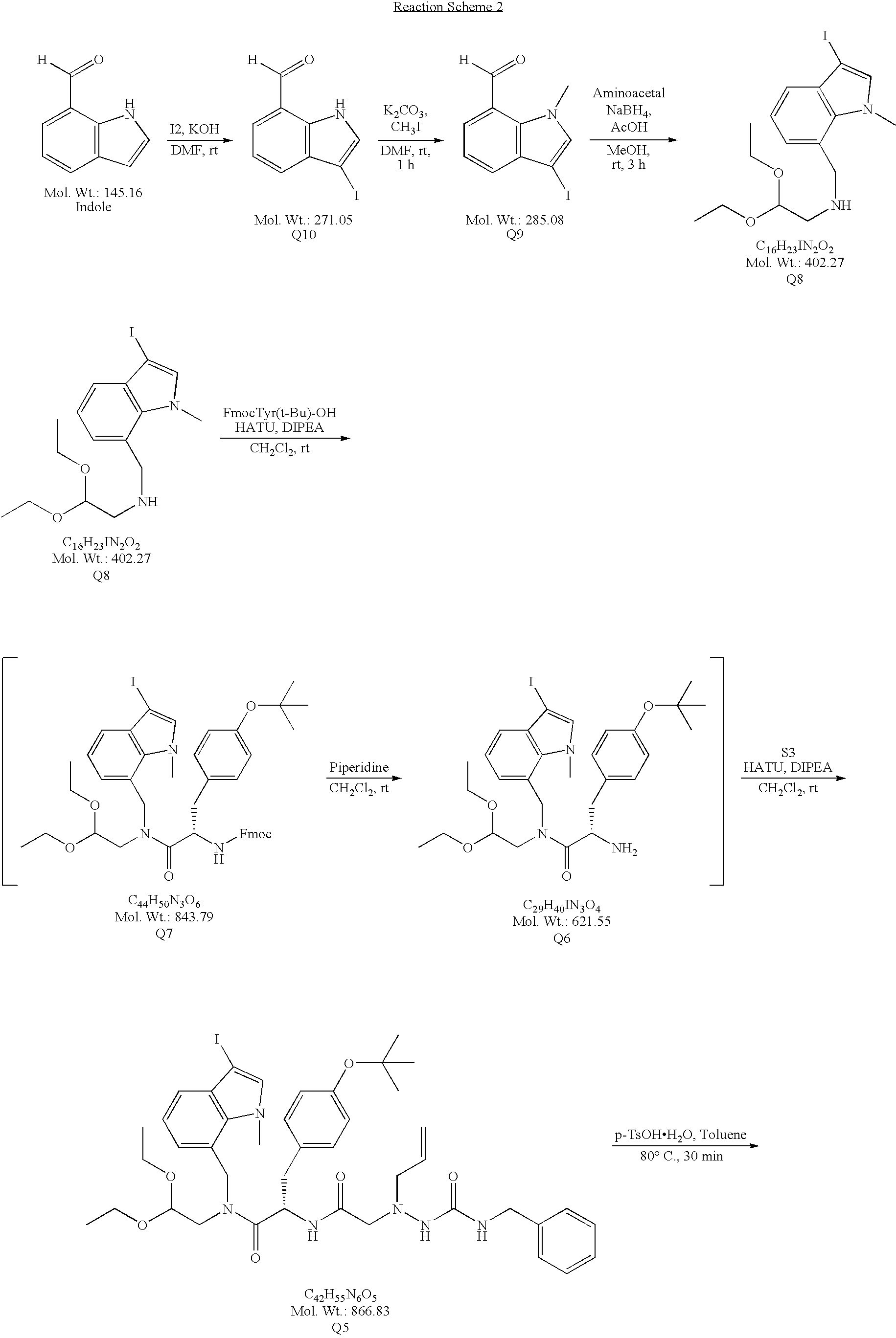

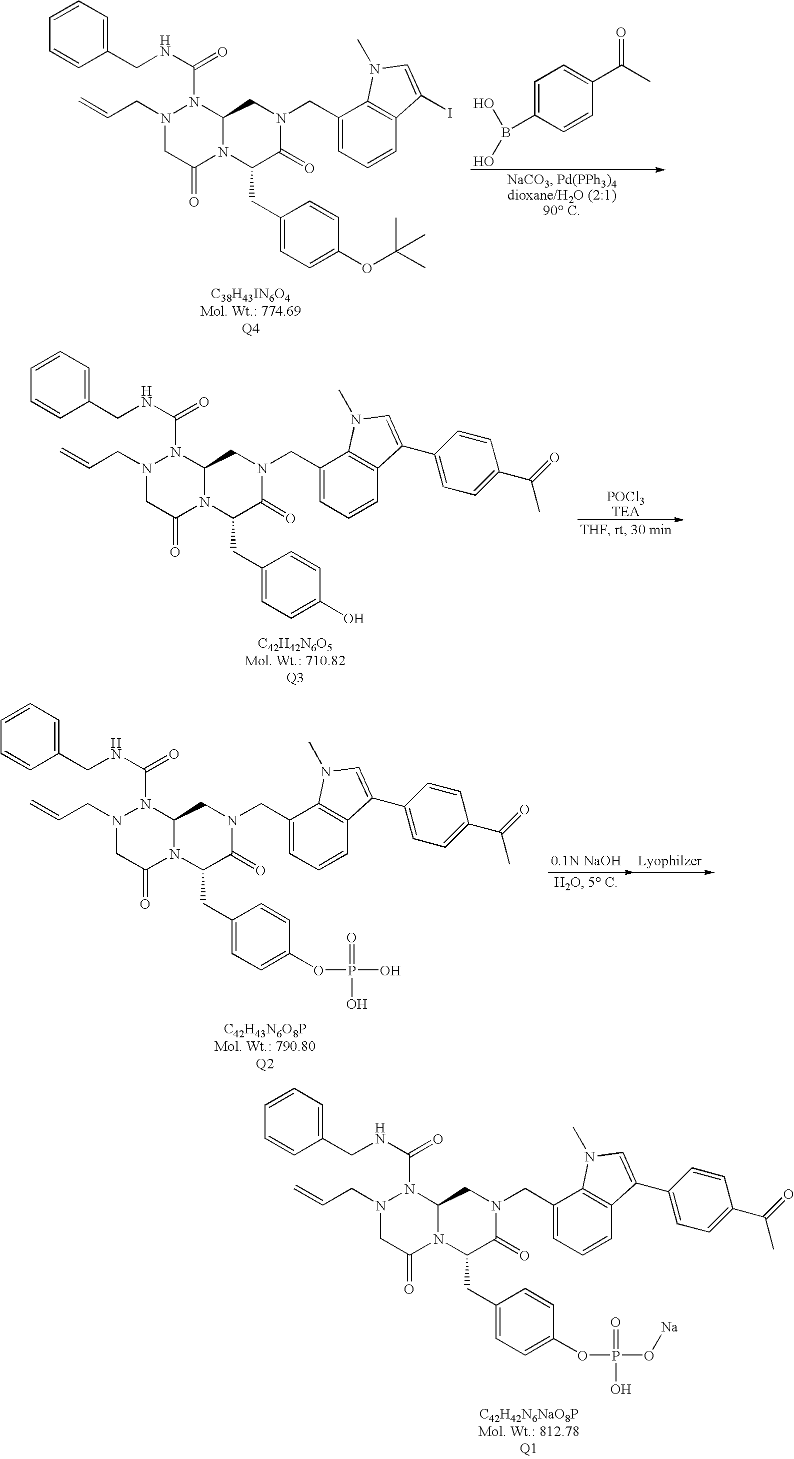

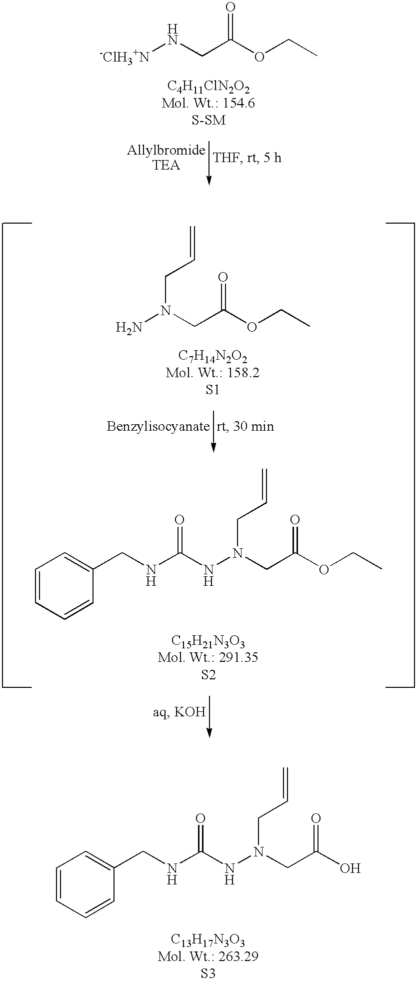

The method illustrated in Reaction Scheme 2 is described in detail in Examples 11 to 21.

### Example 11

### Synthesis of S3 (Side Chain)

S3 was obtained in the same manner as in Example 1.

### Example 12

### Synthesis of Q10

### 3-iodo-1H-indole-7-carbaldehyde

A solution of 24 g of I₂ in 125 ml of DMF (dimethylformamide) was added to the starting material (indole-7-carbaldehyde) and reacted with 5.3 g of KOH with stirring. The reaction progress was monitored with TLC. When the reaction was completed, 354 ml of EA (ethylacetate) and 354 ml of distilled water were added to generate layer separation. The organic layer thus formed was washed with a 10% aqueous Na₂S₂O₃ solution, dried over Na₂SO₄ (sodium sulfate), filtered and concentrated at 40° C. to give Q10 as a concentrated residue.

¹H-NMR (CDCl₃, 300 MHz) δ 10.3 (bs, 1H), 10.2 (s, 1H), 7.79 (d, 1H, J=7.8 Hz), 7.75 (d, 1H, J=7.2 Hz), 7.44 (d, 1H, J=2.1 Hz), 7.37 (t, 1H, J=7.2 Hz); m/z 272.14 [M⁺¹]⁺

### Example 13

### Synthesis of Q9

17 g of Q10 was dissolved in 100 ml of DMF (dimethylformamide) with stirring. The resulting solution was cooled to 10° C. and mixed with 18 g of K₂CO₃ (potassium carbonate). After 6 ml of MeI (methyliodide) was dropwise added thereto, the solution was warmed to room temperature and stirred for 4-5 hrs. When the starting material was recognized as disappearing, K₂CO₃ was filtered off, followed by crystallization in hexane to give Q9.

¹H-NMR (CDCl₃, 300 MHz) δ 10.2 (s, 1H), 7.76 (td, 1H, J=7.8, 1.2 Hz), 7.31 (t, 1H, J=7.8 Hz), 7.12 (s, 1H), 4.14 (s, 3H)

### Example 14

### Synthesis of Q8

To a solution of 18 g of Q9 in 600 ml of MeOH (methanol) was added 0.4 ml of AcOH (acetic acid). At room temperature, 14 ml of aminoacetaldehyde diethylacetal was added to the solution, followed by stirring for 3˜4 hrs. The solution was cooled to 10° C. before 3.3 g of the reducing agent NaCNBH₃ (sodiumcyanoborohydride) was slowly added. At this time, care had to be taken because hydrogen gas and heat were generated. After the reaction mixture was stirred at room temperature for 1 hr, the progress of the reaction was monitored. When the reaction was completed, 354 ml of EA (ethylacetate) and 354 ml of distilled water were used to separate layers. The organic layer thus formed was dehydrated with 141 g of Na₂SO₄ (sodium sulfate) and crystallized in hexane to give Q8.

### Example 15

### Synthesis of Q7

27 g of Fmoc-Tyr(OtBu) was dissolved in 200 ml of MC (dichloromethane) with stirring. To this solution was added 23 g of HATU (O-(7-azabenzotriazol-1-yl)-N,N,N′,N′-tetramethyluronium hexafluorophosphate) and 20 ml of DIPEA (diisopropylethylamine) at room temperature. The solution was stirred for 1˜2 hrs, mixed with 15.8 g of Q9 and further stirred for 2˜3 hrs. After the completion of the reaction, distilled water was added to cause layer separation. The organic layer thus formed was washed with 898 ml of a 5% aqueous citric acid solution and 898 ml of a 5% aqueous NaHCO₃ solution, dehydrated with Na₂SO₄ (sodium sulfate), and concentrated to afford Q7 as a concentrated residue.

### Example 16

### Synthesis of Q6

To a solution of 34 g of Q7 in 400 ml of MC (dichloromethane) was added 20 ml of piperidine. After the reaction is completed, the solution is concentrated, followed by layer separation with 400 ml of MC (dichloromethane) and 800 ml of distilled water. The organic layer thus formed was washed with 850 ml of a 5% aqueous citric acid solution and 850 ml of a 5% aqueous NaHCO₃ solution, and then concentrated to give Q6.

### Example 17

### Synthesis of Q5

To a solution of 13 g of S3 in 400 ml of MC (dichloromethane) were dropwise added 19 g of HATU (O-(7-azabenzotriazol-1-yl)-N,N,N′,N′-tetramethyluronium hexafluorophosphate) and 16 ml of DIPEA (diisopropylethylamine) at room temperature. After the solution was stirred for 2˜3 hrs, a solution of 28 g of Q6 in 200 ml of MC (dichloromethane) was dropwise added thereto. It was stirred at room temperature for 1˜2 hrs. When the reaction was completed, 200 ml of MC (dichloromethane) and 200 ml of distilled water were used to generate layer separation. The organic layer thus formed was washed with 200 ml of a 5% aqueous citric acid solution and 200 ml of a 5% aqueous NaHCO₃ solution and dehydrated with 340 g of Na₂SO₄ (sodium sulfate) and then concentrated to afford Q5 as a concentrated residue.

### Example 18

### Synthesis of Q4

289 mg of p-TsOH.H₂O was added to a solution of 4 g of Q5 in 100 ml of toluene which was then heated to 80° C. The resulting solution was stirred at the same temperature for 30 min, cooled to room temperature and concentrated. Layer separation was generated with EA (ethylacetate) and distilled water. The organic layer was washed with 200 ml of a 5% aqueous citric acid solution and 200 ml of a 5% aqueous NaHCO₃ solution and dehydrated with 340 g of Na₂SO₄ (sodium sulfate) and then concentrated to give Q4 as a concentrated residue.

¹H-NMR (CDCl₃, 300 MHz) δ 7.43˜7.27 (m, 3H), 7.23˜7.21 (m, 2H), 7.12 (t, 1H, J=7.2 Hz), 7.08 (s, 1H), 7.05 (d, 2H, J=7.8 Hz), 6.97 (d, 1H, J=7.2 Hz), 6.90 (d, 2H, J=8.4 Hz), 6.59 (t, 1H, J=6.0 Hz), 5.62 (dd, 1H, J=10.2, 4.8 Hz), 5.53˜5.39 (m, 3H), 5.37 (t, 1H, J=6.0 Hz), 5.02 (d, 1H, J=10.2 Hz), 4.93 (d, 1H, J=16.5 Hz), 4.77 (d, 1H, J=17.1 Hz), 4.44 (dd, 1H, J=15.0, 6.3 Hz), 4.32 (dd, 1H, J=15.0, 6.0 Hz), 3.97 (s, 3H), 3.49˜3.19 (m, 8H), 1.33 (s, 9H);

### Example 19

### Synthesis of Q3

To a solution of 100 mg of Q4 in a mixture of 8 ml of 1,4-dioxane and 4 ml of water were added 33 mg of 4-acetylbenzeneboronic acid, 41 mg of Na₂CO₃ (sodium carbonate) and 15 mg of Pd(PPh₃)₄ (tetrakistriphenylphosphinopalladium), followed by temperature elevation to 90° C. After being stirred for 2 hrs at the same temperature, the solution was cooled to room temperature and concentrated. EA (ethylacetate) and distilled water were used to generate layer separation. The organic layer thus formed was dehydrated with Na₂SO₄ (sodium sulfate) to the concentration. The concentrate was dissolved in MC (dichloromethane) to which 1 ml of TFA (trifluoroacetic acid) was then dropwise added, followed by stirring at room temperature. After the completion of the reaction, the reaction mixture was washed with 10 ml of a 5% aqueous NaHCO₃ solution and dehydrated with Na₂SO₄ (sodium sulfate) to give Q3 as a concentrated residue.

¹H-NMR (CDCl₃, 300 MHz) δ 8.05 (d, 2H, J=8.4 Hz), 7.91 (d, 1H, J=7.2 Hz), 7.71 (d, 2H, J=8.4 Hz), 7.40˜7.20 (m, 4H), 7.16 (t, 1H, J=7.2 Hz), 7.05 (d, 2H, J=8.4 Hz), 6.96 (d, 1H, J=6.9 Hz), 6.69 (d, 2H, J=8.4 Hz), 6.68 (m, 1H), 5.58˜5.44 (m, 3H), 5.37 (t, 1H, J=5.7 Hz), 5.03 (d, 1H, J=10.8 Hz), 4.97 (d, 1H, J=14.7 Hz), 4.81 (d, 1H, J=17.1 Hz), 4.47 (dd, 1H, J=15.3, 6.3 Hz), 4.33 (dd, 1H, J=15.3, 6.3 Hz), 4.33 (s, 3H), 3.47˜3.24 (m, 8H), 2.64 (s, 3H); m/z 711.56 [M⁺¹]⁺

### Example 20

### Synthesis of Q2

A solution of 50 g of Q3 in 217 ml of THF (tetrahydrofuran) was cooled to 0˜5° C. and mixed with 25 ml of POCl₃. At the same temperature, 28 ml of TEA (triethylamine) was dropwise added to the solution which was then stirred for 1 hr. 87 ml of distilled water was slowly added. 348 ml of a sat. aqueous NaHCO₃ solution was added and the solution was stirred for 30 min. The addition of 217 ml of EA (ethylacetate) resulted in layer separation. To the aqueous layer was added 217 ml of MC (methylenechlroride), followed by adjusting the pH of the solution to 1˜3 with 14 ml of conc. HCl. The organic layer thus formed was dehydrated with Na₂SO₄ (sodiumsulfate) and concentrated in a vacuum. The concentrate was crystallized in 130 ml of THF (tetrahydrofuran) and 435 ml of n-hexane and the solid was filtered and dried in a vacuum.

### Example 21

### Synthesis of Q1

44 g of dried Q2 was dissolved in 200 ml of distilled water with stirring. After cooling to 0˜5° C., 0.1 N NaOH was slowly added to adjust the pH of the solution to 4.6˜4.8 (130˜110 mV), followed by lyophilization to give Q1.

A detailed description will be given of the effect of the prepared compounds, below.

### Example 22

The compounds were prepared in the form of prodrugs to improve the solubility thereof. Phosphate may be introduced as a possible prodrug substituent which can exist as in either monosodiumphosphate or disodiumphosphate form.

This prodrug was prepared by adding sodium hydroxide to P2, which was synthesized according to Example 9. Both monosodium and disodium forms of the prodrug show a solubility of up to 400 mg/ml. Both forms have advantageous properties as a composition for I.V. injection in that a monosodium form has pH 4.45 and a disodium form has pH of 7.62.

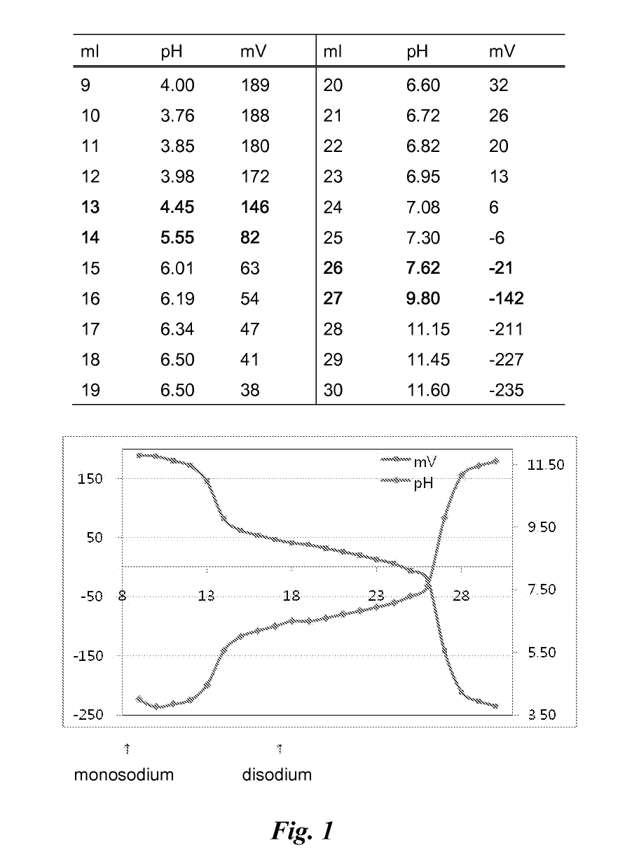

FIG. 1 graphically shows changes in pH and potential when 0.5N NaOH is added dropwise to the compound of the present disclosure. In the graph, the horizontal axis represents the added amounts of sodium hydroxide. In the graph, the first and second points of inflection correspond to the time of production of monosodium and disodium forms, respectively.

### Example 23

### Anticancer Activity in Acute Myeloid Leukemia (AML) Animal Model

Test materials were prepared in the form of prodrugs to increase the solubility of compounds of interest. A phosphate functional group which may be either a monosodium or disodium form was introduced as a prodrug substituent.

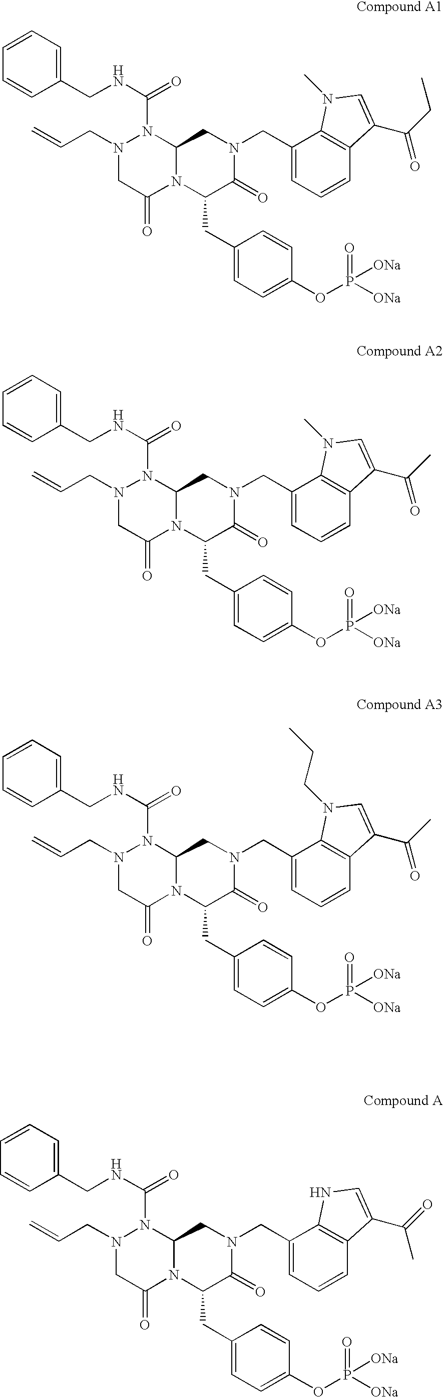

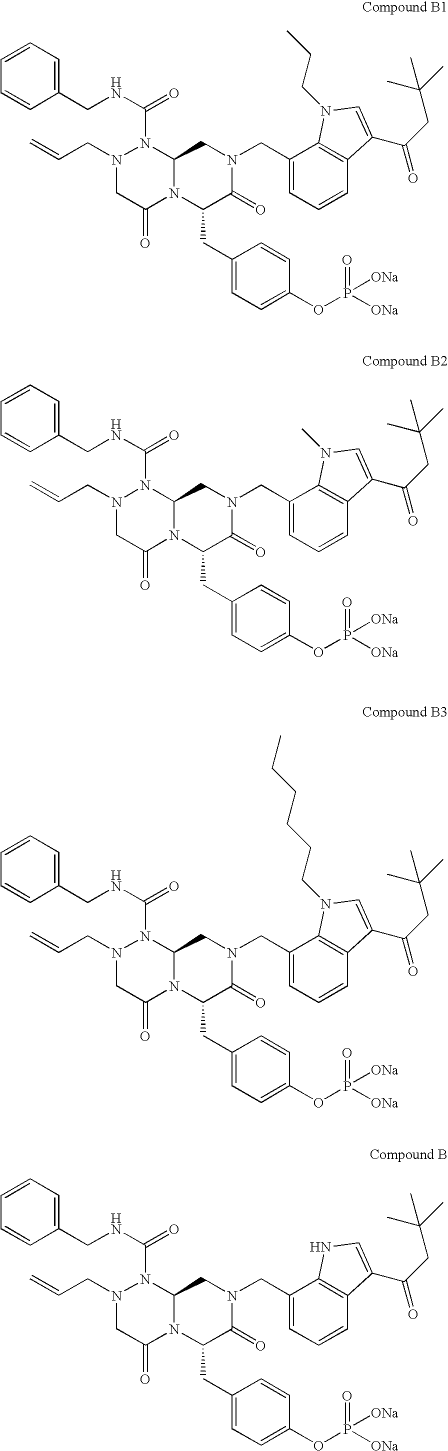

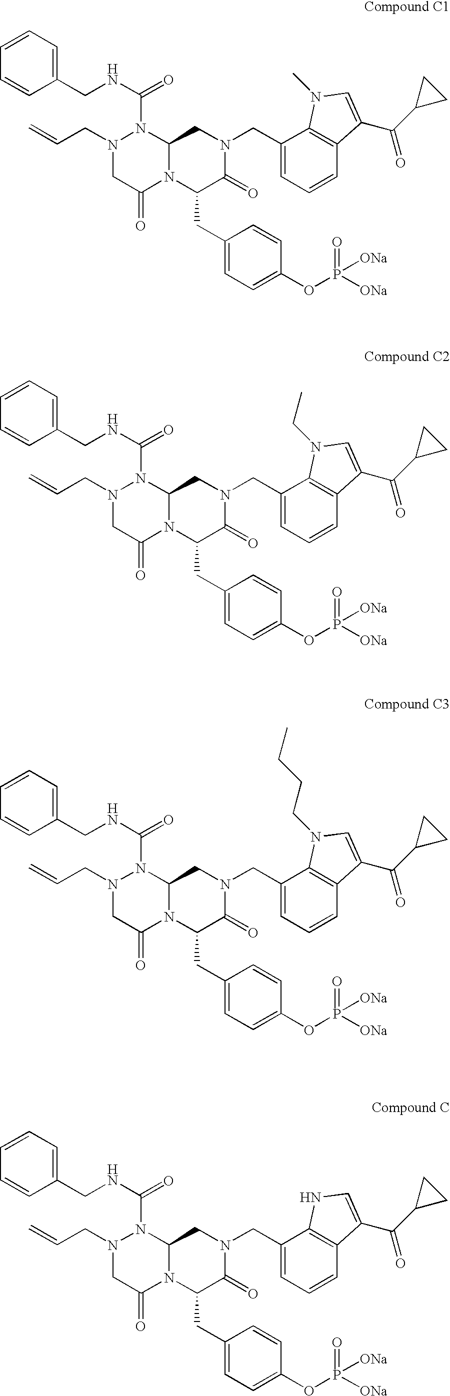

Reference Material: Ara-C (Commercially available drug for treating Acute Myeloid Leukemia)

The human AML cell line, MV4-11, was purchased (ATCC, U.S.A.) and cultured at 37° C. under a 5% CO₂ condition in Iscove's Modified Dulbecco's Medium (GIBCO, cat# 21056) supplemented with 10% fetal bovine serum (GIBCO, cat# 25030-081). Female Balb/C nude mice (OrientBio, Sungnam-city, Korea), 5-6 weeks old, were acclimated to the breeding room. Using a sterilized syringe, a mixture of 1:1 of MV4-11 cells: matrigel (v/v) was implanted in an amount of 5×10⁶/mouse beneath the axilla of each of the mice. When tumor was formed 2 weeks after the implantation, the mice were divided into five (5) groups in such a manner that a minimum deviation with regard to tumor size and body weight was obtained among the groups. The test materials were dissolved in physiological saline and intravenously injected at a dose of 10 ml/kg once a day and five times per week for two weeks (administration days of test materials, D1˜D5, D8˜D12). For a control, only physiological saline was used. The tumor size was determined as calculated by the following equation: Long Axis×Short Axis×Short Axis/2. The Long and Short Axes of the tumor were measured in length using a digital caliper (Mitsutoyo, Japan). The anticancer activity of the test materials was numerated according to the following equation.

Tumor growth Inhibition Rate A (%)=100×[1−(b−a)/(Ref b−Ref a)]

wherein

a=mean tumor size of drug-administered group on Day 1

b=mean tumor size of drug-administered group on Day 12

Ref a=mean tumor size of the control on Day 1

Ref b=mean tumor size of the control on Day 12

When the mean tumor size of the drug-administered group on Day 12 was smaller than that of just before the administration of the test materials, it is indicated as Regression (>100%). Tumor growth Inhibition Rates of tumor growth of the test materials are summarized in Table 2, below.

TABLE 2
INHIBITION RATE OF TUMOR GROWTH

| Test material | dose (mg/kg) | Tumor growth Inhibition Rate |
|---|---|---|
| Ara-C | 50 | 77% |
| Ara-C | 25 | 66% |
| Compound A1 | 25 | Regression (>100%) |
| Compound A2 | 25 | Regression (>100%) |
| Compound A3 | 25 | Regression (>100%) |
| Compound A | 25 | 61% |
| Compound B1 | 25 | Regression (>100%) |
| Compound B2 | 25 | Regression (>100%) |
| Compound B3 | 25 | 80% |
| Compound B | 25 | 49% |
| Compound C1 | 25 | Regression (>100%) |
| Compound C2 | 25 | Regression (>100%) |
| Compound C3 | 25 | 70% |
| Compound C | 25 | 25% |

Test results exhibit that all test compounds have inhibitory activity against tumor growth. In compounds A1-A3, B1-B3 and C1-C3 according to the present disclosure, tumor inhibition rates were measured to range from 70% to regression (>100%). In contrast, Ara-C, a widely used drug for AML, was found to have a tumor inhibition rate of 66%. Taken together, the results demonstrate that the compounds of the present disclosure are highly inhibitory of tumor growth.

### Example 24

### In Vitro Cardiotoxicity Assay: Assay for Inhibitory Activity Against hERG

HEK293 was transfected with hERG (human Ether-à-go-go Related Gene) cDNA for 48 hrs using Lipofectamine 2000 (Invitrogen, USA). The transfected HEK293 cells were cultured in Modified Dulbecco's Medium (MEM, Gibco, 1 L) supplemented with 10% FBS, sodium pyruvate (10 ml), penicillin/streptomycin (10 ml) and Zeocin (100 μg/ml, Invitrogen) at 37° C. under 5% CO₂. After being detached from incubation vessels by trypsinization, the HEK293 cells were placed in a chamber for patch clamp recording. A whole-cell patch clamp method was used to record hERG K+ currents in HEK293 cells using the following intra/extracellular solutions. Thereafter, Effects on K+ currents were observed with the compounds applied outside the cells.
intracellular solution: K-aspartate 100 mM, KCl 25 mM, NaCl 5 mM, MgCl₂ 1 mM, Mg-ATP 4 mM, 1,2-bis(o-aminophenoxy)ethane-N,N,N′,N′-tetraacetic acid (BAPTA) 10 mM, 4-(2-hydroxyethyl)-1-piperazineethanesulfonic acid (HEPES) 10 mM, normalized magnesium (NMG) were used to adjust the pH to 7.2;
extracellular solution: NaCl 145 mM, KCl 5 mM, glucose 10 mM, MgCl₂ 1 mM, CaCl₂ 2 mM, HEPES10 mM, HCl were used to adjust the pH to 7.4.

The membrane potential was depolarized from −80 mV to +20 mV for 1,000 ms in a whole-cell patch clamp mode and then repolarized to −40 mV for 1,000 ms, during which the tail current of outward hERG K+ currents was recorded. In this regard, the concentrations of the compounds that are required for 50% inhibition of the current were represented as IC₅₀.

TABLE 3
CARDIOTOXICITY ASSAY

| | Cardiotoxicity (μM) |
|---|---|
| Test Cpd. | (hERG Inhibiting Activity Assay, IC₅₀) |
| Compound A1 | 80 |
| Compound A | 14 |
| Compound B1 | 18 |
| Compound B2 | 25 |
| Compound B3 | 20 |
| Compound B | 1.6 |

The risk of cardiotoxicity has been raised in many drugs. Some of them were withdrawn from the market because they caused a sudden death due to the cardiotoxicity thereof. The cardiotoxicity of drugs is associated with the extension of QT intervals on electrocardiograms. Particularly, most of the drugs extending QT intervals are known to inhibit IKr channels (Bernard Fermini and Anthony A. Fossa, Nature Reviews Drug Discovery, 2003, 2, 439-447). The hERG channel shows the most important effect on cardiotoxicity among IKr channels. In this example, the risk of cardiotoxicity was evaluated using human hERG channel-expressing mammal cells, which are internationally recognized as a system (ICH guideline, S7B, Step 4, 12, May, 2005). Although pharmaceutical activity of drug should be taken into consideration, a drug is evaluated as having a low cardiotoxicity risk when IC₅₀ thereof is 10 μM or higher. In this assay, most test compounds were found to overpass this criterion. Having higher IC₅₀, compound A1 was evaluated to be safer than compound A, and compounds B1, B2 and B3 than compound B.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound of Chemical Formula I: 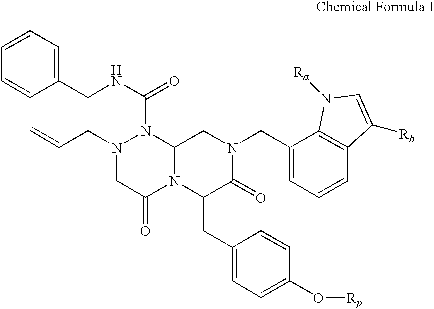
wherein:
Rₐ is a C₁-C₆ alkyl group, a C₂-C₆ alkenyl, or a C₂-C₆ alkynyl group;
R_{b} is an aryl group, a substituted aryl group, or —C(═O)Rₑ, wherein Rₑ is a C₁-C₆ alkyl group, a C₂-C₆ alkenyl group, or a C₂-C₆ alkynyl group; and
Rₚ is —H, —PO₃H₂, —HPO₃⁻Na⁺, —PO₃²⁻Na₂⁺, —PO₃²⁻K₂⁺, —PO₃²⁻Mg²⁺, —PO₃²⁻Ca²⁺,

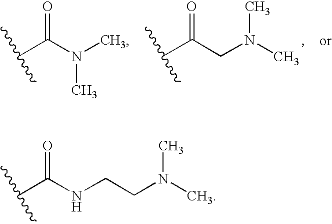

2. The compound according to claim 1, wherein:
Rₐ is a C₁-C₆ alkyl group or a C₂-C₆ alkenyl group,
R_{b} is —C(═O)Rₑ wherein Rₑ is C₁-C₆ alkyl, and
Rₚ is —H, —PO₃H₂, —HPO₃⁻Na⁺, or —PO₃²⁻Na₂⁺.

3. The compound according to claim 1, wherein
Rₐ is methyl,
R_{b} is —C(═O)Rₑ wherein Rₑ is C₁-C₆ alkyl, and
Rₚ is —PO₃H₂, —HPO₃⁻Na⁺, or —PO₃²⁻Na₂⁺.

4. The compound according to claim 1, wherein the substituted aryl is acyl-substituted aryl.

5. The compound according to claim 1, wherein the compound represented by Chemical Formula I is
2-Allyl-8-[3-(3,3-dimethyl-butyryl)-1-methyl-1H-indol-7-ylmethyl]-6-(4-hydroxy-benzyl)-4,7-dioxo-hexahydro-pyrazino[2,1-c][1,2,4]triazine-1-carboxylic acid benzylamide,
2-Allyl-6-(4-hydroxy-benzyl)-8-[1-methyl-3-(3-methyl-butyryl)-1H-indol-7-ylmethyl]-4,7-dioxo-hexahydro-pyrazino[2,1-c][1,2,4]triazine-1-carboxylic acid benzylamide,
2-Allyl-8-(3-butyryl-1-methyl-1H-indol-7-ylmethyl)-6-(4-hydroxy-benzyl)-4,7-dioxo-hexahydro-pyrazino[2,1-c][1,2,4]triazine-1-carboxylic acid benzylamide,
2-Allyl-8-(3-cyclopropanecarbonyl-1-ethyl-1H-indol-7-ylmethyl)-6-(4-hydroxy-benzyl)-4,7-dioxo-hexahydro-pyrazino[2,1-c][1,2,4]triazine-1-carboxylic acid benzylamide,
2-Allyl-8-(1-allyl-3-cyclopropanecarbonyl-1H-indol-7-ylmethyl)-6-(4-hydroxy-benzyl)-4,7-dioxo-hexahydro-pyrazino[2,1-c][1,2,4]triazine-1-carboxylic acid benzylamide,
2-Allyl-6-(4-hydroxy-benzyl)-8-(1-methyl-3-pentanoyl-1H-indol-7-ylmethyl)-4,7-dioxo-hexahydro-pyrazino[2,1-c][1,2,4]triazine-1-carboxylic acid benzylamide,
2-Allyl-6-(4-hydroxy-benzyl)-8-(1-methyl-3-propionyl-1H-indol-7-ylmethyl)-4,7-dioxo-hexahydro-pyrazino[2,1-c][1,2,4]triazine-1-carboxylic acid benzylamide,
8-(3-Acetyl-1-propyl-1H-indol-7-ylmethyl)-2-allyl-6-(4-hydroxy-benzyl)-4,7-dioxo-hexahydro-pyrazino[2,1-c][1,2,4]triazine-1-carboxylic acid benzylamide,
2-Allyl-8-[3-(3,3-dimethyl-butyryl)-1-propyl-1H-indol-7-ylmethyl]-6-(4-hydroxy-benzyl)-4,7-dioxo-hexahydro-pyrazino[2,1-c][1,2,4]triazine-1-carboxylic acid benzylamide,
2-Allyl-8-[3-(3,3-dimethyl-butyryl)-1-hexyl-1H-indol-7-ylmethyl]-6-(4-hydroxy-benzyl)-4,7-dioxo-hexahydro-pyrazino[2,1-c][1,2,4]triazine-1-carboxylic acid benzylamide, or
2-Allyl-8-(1-butyl-3-cyclopropanecarbonyl-1H-indol-7-ylmethyl)-6-(4-hydroxy-benzyl)-4,7-dioxo-hexahydro-pyrazino[2,1-c][1,2,4]triazine-1-carboxylic acid benzylamide.

6. A pharmaceutical composition comprising the compound according to claim 1 and a pharmaceutically acceptable excipient.

7. A method of treating acute myeloid leukemia (AML) comprising administering to a patient having AML an effective amount of the pharmaceutical composition according to claim 6.

8. The method of claim 7 wherein administering comprising injecting the pharmaceutical composition to the patient.

9. A method for manufacturing the compound of claim 1, comprising the following sequential steps:
introducing an acyl group into indole-7-carbaldehyde through Friedel-Crafts Acylation to provide 3-acyl-indole-7-carbaldehyde;
introducing an alkyl group and an aminoacetal group to 3-acyl-indole-7-carbaldehyde to provide a 1-alkyl-3-acyl-indole derivative;
amidating the 1-alkyl-3-acyl-indole derivative with stereoselectivity Cbz-Tyrosine-OtBu and 2-(1-allyl-4-benzylsemicarbazido)acetic acid to provide a reaction intermediate;
cyclizing the reaction intermediate in the presence of formic acid to provide a cyclic intermediate; and
phosphorylating the cyclic intermediate to provide a compound of Chemical Formula (I).

10. The method according to claim 9, wherein 2-(1-allyl-4-benzylsemicarbazido)acetic acid is synthesized by the following sequential steps:
adding TEA (triethylamine) to an ethylhydrazinoacetate solution to provide a reaction solution;
adding allyl bromide to the reaction solution; and then adding benzylisocyanate.

11. The method of claim 10 wherein allyl bromide and benzylisocyanate are added in a dropwise manner.

12. A method for preparing a compound of Chemical Formula (I), comprising:
converting indole-7-carbaldehyde to 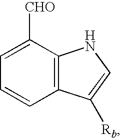
wherein R_{b} is an aryl group, a substituted aryl group, or —C(═O)Rₑ, wherein Rₑ is a C₁-C₆ alkyl group, a C₂-C₆ alkenyl group, or a C₂-C₆ alkynyl group;
converting 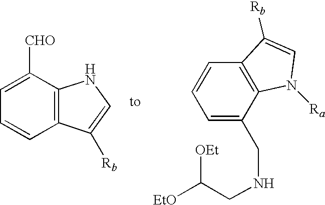
wherein Rₐ is a C₁-C₆ alkyl group, a C₂-C₆ alkenyl, or a C₂-C₆ alkynyl group;
amidating 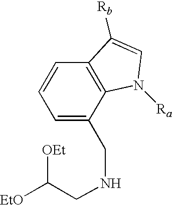
with stereoselectivity in the presence of Cbz-Tyrosine-OtBu and 2-(1-allyl-4-benzylsemicarbazido)acetic acid to provide 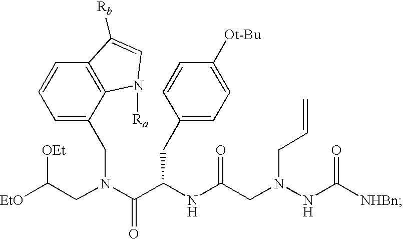
cyclizing 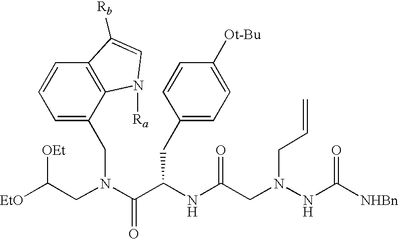
in the presence of formic acid to provide 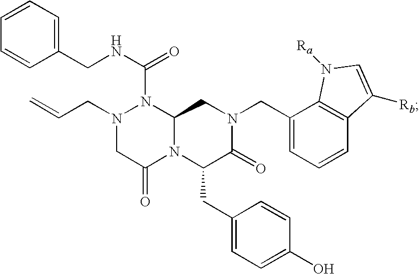
converting 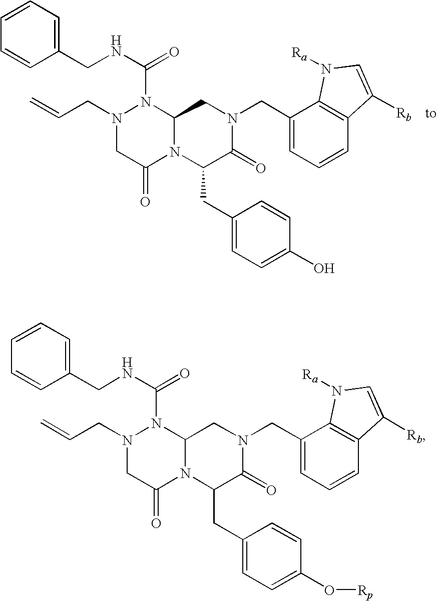
wherein Rₚ is —PO₃H₂, —HPO₃⁻Na⁺, —PO₃²⁻Na₂⁺, —PO₃²⁻K₂⁺, —PO₃²⁻Mg²⁺, —PO₃²⁻Ca²⁺.

13. The method of claim 12, where Rₐ is methyl, Rb is —C(═O)Rₑ, and Rₑ is methyl or cyclopropyl.
